(19) United States
(12) Patent Application Publication
(10) Pub. No.: US 2011/0123612 A1
(43) Pub. Date: May 26, 2011

(54) PHARMACEUTICAL PREPARATION CONTAINING NON-DIHYDROPYRIDINE CALCIUM CHANNEL BLOCKER AND ANGIOTENSIN-2 RECEPTOR BLOCKER

(76) Inventors: Sung Wuk Kim, Gyeonggi-do (KR); Sung Soo Jun, Gyeonggi-do (KR); Ah Ram Lee, Incheon (KR); Sang Ouk Sun, Gwangju (KR); Jin Won Choi, Gyeonggi-do (KR); Young Gwan Jo, Daejeon (KR); Ja Seong Koo, Daejeon (KR)

(21) Appl. No.: 12/736,442

(22) PCT Filed: Apr. 3, 2009
(86) PCT No.: PCT/KR2009/001723

§ 371 Date: Dec. 9, 2010

(30) Foreign Application Priority Data
Apr. 10, 2008 (KR) 10-2008-0033364

(51) Int. Cl. A61K 9/52; A61K 9/00; A61K 9/22; A61K 9/28; A61K 31/554; A61K 31/4178; A61P 9/12; A61P 9/00

(52) U.S. Cl. 424/457; 424/400; 424/471; 424/472; 514/211.06; 514/381

(57) ABSTRACT

The present invention provides a pharmaceutical formulation comprising an immediate-release compartment containing an angiotensin-2 receptor blocker (ARB) as a pharmacologically active ingredient and an extended-release compartment containing a non-dihydropyridine calcium channel blocker as a pharmacologically active ingredient. Since the disclosed formulation enables the release of the two ingredients at a different time, it reduces side effects and increases the effects of the drug more than the case of separately administering the ingredients each at the same time. In addition, the formulation maximizes the effects of drug at the time of day when the complication risk of cardiovascular system diseases is highest.

### TECHNICAL FIELD

The present invention relates to a pharmaceutical formulation containing a non-dihydropyridine calcium channel blocker and an angiotensin-2 receptor blocker (ARB) such as losartan.

### BACKGROUND ART

Anti-hypertensive therapy aims not only to lower blood pressure but also to prevent complications such as myocardial infarction, cardiac insufficiency, cerebral stroke and early death, which are likely to arise in hypertensive patients, or to prevent the condition of the diseases from becoming worse, thereby securing long and healthy lives of those patients.

There are various causes for hypertension. Thus, it is difficult to predict what results are shown when a single anti-hypertensive agent is used (Journal of many hypertension 1995: 9: S33-S36). For this reason, a combination therapy of anti-hypertensive agents has been continuously increased. A combination therapy of anti-hypertensive agents has been reported as necessary for the following reasons in the clinical and academic fields [J. Hum. Hypertens. 1995: S33-S36].

1) Hypertension is caused by multiple causes and factors even in the same patients.

2) It is natural that a single drug cannot treat multiple pathophysiological conditions of diseases.

3) A single drug is only effective to less than 50% of patients.

4) A combination prescription is effective to more than 80% of patients.

5) In particular, by prescribing only a single drug, it is difficult to attain desired anti-hypertensive effects against hypertension of patients with complications such as diabetes, and even more difficult to prevent the occurrence of complications.

6) When the dose is increased because a single drug at a low dose is not satisfactory in efficacy, side effects may be increased.

7) A combination prescription of drugs having different pharmacological effects may remove the various causes of diseases while preventing complications and reducing side effects. Therefore, the American Heart Association also emphasizes that the best way is to start with a combination prescription of drugs rather than starting with a single drug prescription.

8) In particular, it is even more important that blood pressure should be significantly lowered in patients with complications than those without. A combined therapy is essential in this case. Nevertheless, a single drug may be effective only for 26% of patients. A combination formulation may be effective for as many as 74% of patients in preventing complications by maintaining proper blood pressure [Large-scaled clinical test, HOT].

9) The US FDA has been acknowledging for 30 years the necessity of a combined formulation in accordance with a fixed-dose combination therapy. According to this principle, it is required that drugs with different pharmacological activities be combined to contain the same amounts, as if they were taken in each single drug. This is called a fixed-dose combination formulation, and has been approved without further experimental data if the efficacy and safety of the single drugs are fully evaluated and such combination therapy has been widely prescribed by doctors.

10) It is well known that a fixed-dose combination of anti-hypertensive agents has excellent effects in lowering blood pressure. The dose of each ingredient is not increased, and thus the side effects of each ingredient are greatly reduced.

11) Anti-hypertensive agents cause side effects mainly related to the circulatory system. Accordingly, ingredients having different pharmacological activity are frequently combined with each other to offset side effects thereof.

12) A combined pharmaceutical formulation may improve medication compliance, and save a half of the time required for doctors to educate aged patients regarding medication instruction.

13) A combination therapy formulation may lower the risk of circulatory complications, and reduce the long-term expenses to be incurred for disease control.

In general, blood pressure increases with age. About 63% of elderly people above the age of 60 suffer from hypertension. Due to declined renal functions, such geriatric hypertensive patients generally exhibit increased production of angiotensin which is a blood pressure-raising factor, thereby further elevating blood pressure. In this case, with the use of a single drug, it is difficult to lower systolic hypertension to a desired level. Therefore, a combination therapy of losartan, which is one of ARBs (Angiotensin-2 receptor blockers) effective in protection of the kidney, and amlodipine, which is a calcium channel blocker, has been typically adopted as an effective medication [Clin. Ther. 2003 May 25 (5): 1469-69; Nepherol Dial Transplant vol. 18 (2003): p 1806-1813; J. American Society of Nephrology, vol. 12 (2001) p. 822-827]. Pharmacological activities of amlodipine and losartan ingredients are as follows.

TABLE 1

| | Amlodipine | Losartan |
|---|---|---|
| 1) Vasodilation lowering a | Relaxes blood vessels by | Relaxes blood vessels by |
| blood pressure | decreasing an inflow of | blocking activity of |
| | calcium ions into blood | vasoconstriction factors and |
| | vessel wall smooth muscle | suppressing activity of |
| | cells | aldosterone that increases a |
| | | blood pressure-raising |
| | | substance angiotensin-2 |
| 2) Circadian Biorhythmic | Favorably effective in stress- | Strong post-midnight anti- |
| action according to drugs | induced vasospasm during | hypertensive effects by |
| | the morning after rising | suppressing excitation of |
| | | RAAS* system during post- |
| | | midnight sleeping and more |
| | | favorably effective in non- |
| | | dipper hypertension |
| 3) Influence on electrolyte | Causes the loss of K⁺ | Suppresses the loss of K⁺ |
| 4) Action on renin | Very effective in patients | Very effective in patients |
| | with hyporeninism | with hyperreninism |
| 5) Glucose metabolism | Favorably acting on diabetic | Helpful in diabetic patients |
| | patients by improving | by increasing insulin |
| | glucose metabolism | sensitivity |
| | Reducing insulinemia | |
| 6) inhibiting deterioration of | Suppressing the atheromic | Inhibiting the proliferation of |
| atherosclerosis and | proliferation in blood vessel | pathogenic cells by |
| hyperlipidemia causing | walls | suppressing the stimulus of |
| complications | Suppressing the | angiotensin-2 on blood vessel |
| | overproduction of connective | walls |
| | tissues in blood vessel walls | |
| | Suppressing the | |
| | peroxidation of LDLs | |
| | Lowering plasma | |
| | triglycerides | |
| 7) Vascular endothelial | Stimulating the regeneration | Stimulating the regeneration |
| function | and maintenance of vascular | and maintenance of vascular |
| | endothelial cells | endothelial cells |
| 8) Vasodilating effects on | Acting on afferent vessels | Acting on efferent vessels |
| glomerular artery | | |
| 9) Albuminuria | Lowered | Lowered |
| 10) Proliferation of kidney | Suppressed | Suppressed |
| mesangial cells | | |

*RASS (Renin and Angiotensin System): One of mechanisms for regulating blood pressure in vivo
*Non-dipper hypertension: Hypertension where blood pressure does not decrease in one's sleep unlike normal people. Frequently found in aged people and patients suffering from diabetes or cardiac hypertrophy. Relatively high risk of complications such as cerebral stroke.

That is, when the two drugs are administered at certain time intervals, a combination therapy of the two drugs may show a synergistic effect in controlling hypertension for a patient, to whom the efficacy of single drug is insufficient, because the two drugs are different from each other in the mechanism in lowering blood pressure and the time of maximum activity. Further, it can be said that the two drugs in combination prescription are complementary to each other in that amlodipine induces loss of potassium ions whereas losartan inhibits loss of potassium ions.

Meanwhile, diltiazem, a representative one of the non-dihydropyridine calcium channel blocker drugs, is a benzothiazepine drug and is metabolized via N-demethylation by the action of cytochrome P450 (CYP450) in the liver. Among the resulting metabolites of diltiazem, N-desmethyl diltiazem and N,N-didesmethyl diltiazem exhibit more potent inhibitory effects on the synthesis of cytochrome P450 3A4 than diltiazem. Such inhibitory effects last during diltiazem therapy [Br. J. Clin. Pharmacol. 1997; 282: 294-300]. Inhibitory effects of these metabolites on cytochrome P450 3A4 result from the formation of a metabolite intermediate complex between N-desmethyl diltiazem and cytochrome P450 3A4 expressed by a cDNA in the liver microsome [J. Pharmacol. Exp. Ther. 1999, 290, 1116-1125]. Thereby, diltiazem irreversibly inhibits cytochrome P450 3A4, irrespective of the cytochrome P450 3A4 protein or mRNA level, unlike an increase in the level of a cytochrome P450 3A4 protein upon long-term therapeutic medication of erythromycin or troleandomycin which is a representative cytochrome P450 inhibitor [Br. J. Clin. Pharmacol. 2005; 59(4):440 to 446]. Accordingly, these inhibitory effects of diltiazem may have an influence on effects of losartan whose active form has 10-fold or more anti-hypertensive effects, or on effects of other ARB drugs.

Verapamil, another representative non-dihydropyridine calcium channel blocker drug, is a phenylalkylamine drug and is classified as a cytochrome P450 3A4 inhibitor. This drug may also decrease effects of ARB drugs upon co-administration therewith.

Further, losartan, a representative prescription example of ARB, is a drug which has anti-hypertensive effects and also exhibits broad effects such as prevention and treatment of heart failure, prevention and treatment of post-myocardial infarction arrhythmia and heart failure, prevention and treatment of diabetic complications, prevention and treatment of renal failure, prevention and treatment of cerebral stroke, anti-platelet effects, arteriosclerosis-preventive effects, suppression of harmful effects of aldosterone, reduction of effects of metabolic syndromes, and effects of preventing circulatory diseases from becoming worse in a chain-like manner [Clin, Exp. Hypertens., vol. 20 (1998), p. 205-221, J. Hypertens., vol. 13 (8) (1995), p. 891-899, Kidney Int., vol. 57(2) (2000), p. 601-606, Am. J. Hypertens., vol. 10 (12PT2) Suppl. (1997), p. 325-331, Circulation, vol. 101(14) (2000), p. 1653-1659, J. Hypertension., vol 17 (7) (1999), p. 907-716, Circulation, vol. 101 (2000), p. 2349]. Losartan is absorbed and transported to the liver. Some of losartan is released into blood in the form of an active losartan molecule, reaching a peak blood concentration within 1 hour. However, the remaining portion of losartan is metabolized by two hepatic enzymes, cytochrome P450 2C7 and 3A4 and is therefore converted into losartan carboxylic acid having a higher activity, reaching the highest blood concentration after 3 to 4 hours. That is, the total pharmacological activity of losartan is the pharmacological action of a mixture of losartan and losartan carboxylic acid (losartan's active metabolite).

As discussed above, the two anti-hypertensive agents act with different time of maximum activity, and it is known by the xenobiotics that the two drugs may be affected by or act on the same enzyme in an opposite manner when released and absorbed in the liver at the same time [Cytochrome P450 Drug Interaction Table, Department of Medicine, Indiana University updated 2004 Mar. 11].

In this respect, the following problems may be caused when a non-dihydropyridine drug and an ARB drug are co-administered.

Non-dihydropyridine calcium channel blocker drugs inhibit a cytochrome P450 3A4 enzyme. ARB drugs are affected by CYP 3A4. Accordingly, if a non-dihydropyridine drug and an ARB drug are absorbed at the same time, expression of ARB drug efficacy will be decreased. In particular, isolated systolic blood pressure in aged people may cause cerebral stroke, even though such isolated systolic blood pressure is slightly out of control. Therefore, blood pressure should be maintained at the normal level strictly for 24 hours to prevent complications.

However, no pharmaceutical formulation that may overcome the aforementioned clinical drawbacks has been reported.

U.S. Pat. Nos. 5,721,244 and 6,677,356 disclose a pharmaceutical formulation of an angiotensin converting enzyme inhibitor and a calcium channel blocker, and an anti-hypertensive drug which can be administered for the treatment of cardiovascular diseases, respectively. However, the above-stated inventions are completely different from a chronotherapeutic pharmaceutical formulation of a non-dihydropyridine calcium channel blocker and an ARB drug which is sought by the present invention. Further, a pharmaceutical formulation of two different drugs is not yet commercially available.

Korean Patent Application Laid-Open Publication No. 2004-0078140 discloses a use of a valsartan/calcium channel blocker pharmaceutical formulation. However, the above-stated invention relates to a simple combination formulation which is intended for the treatment of hypertension upon simple combination therapy of two drugs and is therefore different from the present invention which is intended for the development of a pharmaceutical formulation exhibiting excellent anti-hypertensive effects by the use of a functional combination formulation technique.

Korean Patent No. 0222627 discloses a novel composition of ARB drug/calcium channel blocker. This patent is an invention relating to the synthesis in which two main ingredients are combined to be administered as one ingredient and is therefore totally different from the present invention.

### DISCLOSURE OF THE INVENTION

### Technical Problem

To overcome the aforementioned problems, the present inventors have exerted extensive research and finally developed a combination drug system and a functional pharmaceutical formulation where an ARB drug such as losartan is absorbed in the small intestine immediately after the administration while a non-dihydropyridine calcium channel blocker such as diltiazem or verapamil is absorbed in the small intestine 2 to 4 hours after the administration, thereby it is possible to maintain constant blood pressure for 24 hours and inhibit complications and other side effects by the administration once a day in the evening, and thus completed the present invention.

Therefore, the present invention is intended to provide a combination drug system and a functionally designed pharmaceutical formulation that can maximize the pharmacological and clinical efficacy in the treatment of hypertension and in the prevention of its complications or other side effects, as compared to the co-administration of a non-dihydropyridine calcium channel blocker single drug and an ARB single drug.

### Technical Solution

The present invention provides a pharmaceutical formulation including a prior-release compartment containing an angiotensin-2 receptor blocker (ARB) as a pharmacologically active ingredient and a delayed-release compartment containing a non-dihydropyridine calcium channel blocker as a pharmacologically active ingredient.

In the present invention, ARB is preferably at least one selected from losartan, valsartan, telmisartan, eprosartan, irbesartan, candesartan, olmesartan, and if any, isomers thereof, pharmaceutically acceptable salts thereof, and prodrugs thereof.

In the present invention, the non-dihydropyridine calcium channel blocker refers to a pharmaceutical formulation which is a non-dihydropyridine calcium channel blocker which inhibits the production of a cytochrome P450 enzyme, and for example, may be at least one selected from diltiazem, verapamil, gallopamil, cinnarizine, flunarizine, isomers thereof and pharmaceutically acceptable salts thereof. The non-dihydropyridine calcium channel blocker is more preferably at least one selected from diltiazem, verapamil, isomers thereof and pharmaceutically acceptable salts thereof.

The present invention provides a pharmaceutical formulation wherein ARB is losartan or a pharmaceutically acceptable salt thereof, and the non-dihydropyridine calcium channel blocker is diltiazem, an isomer thereof or a pharmaceutically acceptable salt thereof.

The present invention provides a pharmaceutical formulation wherein ARB is valsartan, an isomer thereof or a pharmaceutically acceptable salt thereof, and the non-dihydropyridine calcium channel blocker is diltiazem, an isomer thereof or a pharmaceutically acceptable salt thereof.

The present invention provides a pharmaceutical formulation wherein ARB is telmisartan or a pharmaceutically acceptable salt thereof, and the non-dihydropyridine calcium channel blocker is diltiazem, an isomer thereof or a pharmaceutically acceptable salt thereof.

The present invention provides a pharmaceutical formulation wherein ARB is candesartan, a pharmaceutically acceptable salt thereof or a prodrug thereof, and the non-dihydropyridine calcium channel blocker is diltiazem, an isomer thereof or a pharmaceutically acceptable salt thereof. The candesartan prodrug is decomposed in vivo into candesartan and is preferably candesartan cilexetil.

The present invention provides a pharmaceutical formulation wherein ARB is irbesartan or a pharmaceutically acceptable salt thereof, and the non-dihydropyridine calcium channel blocker is diltiazem, an isomer thereof or a pharmaceutically acceptable salt thereof.

The present invention provides a pharmaceutical formulation wherein ARB is losartan or a pharmaceutically acceptable salt thereof, and the non-dihydropyridine calcium channel blocker is verapamil, an isomer thereof or a pharmaceutically acceptable salt thereof.

The present invention provides a pharmaceutical formulation wherein ARB is olmesartan, a pharmaceutically acceptable salt thereof or a prodrug thereof, and the non-dihydropyridine calcium channel blocker is diltiazem, an isomer thereof or a pharmaceutically acceptable salt thereof. The olmesartan prodrug is decomposed in vivo into an active ingredient olmesartan and is preferably olmesartan medoxomil.

The present invention provides a pharmaceutical formulation wherein ARB is eprosartan or a pharmaceutically acceptable salt thereof, and the non-dihydropyridine calcium channel blocker is diltiazem, an isomer thereof or a pharmaceutically acceptable salt thereof.

Hereinafter, unless otherwise specified, referring to a pharmacologically active ingredient name is intended to include an isomer thereof and a pharmaceutically acceptable salt thereof. For example, even though “losartan” may be described hereinafter, this term is construed to include an isomer of losartan and a pharmaceutically acceptable salt of losartan, if any.

The present invention provides a pharmaceutical formulation wherein ARB is released at a level of more than 60% of a total amount of ARB in the formulation within one hour after initiation of the release thereof. Preferably, ARB is released at a level of more than 85% of a total amount of ARB in the formulation within 30 minutes after initiation of the release thereof.

Further, the present invention provides a pharmaceutical formulation wherein the release of the non-dihydropyridine calcium channel blocker is initiated 2 hours after the release of ARB is initiated, and is finished within 24 hours.

Further, the present invention provides a pharmaceutical formulation wherein the non-dihydropyridine calcium channel blocker is released at a level of less than 60% of a total amount of a non-dihydropyridine calcium channel blocker in a unit formulation up to 4 hours after the release of ARB is initiated.

In the present invention, examples of the pharmaceutically acceptable salt include inorganic ion salts formed with calcium, potassium, sodium, magnesium, etc., inorganic acid salts formed with hydrochloric acid, nitric acid, phosphoric acid, bromic acid, iodic acid, perchloric acid, tartaric acid, sulfuric acid, etc.; organic acid salts formed with methanesulfonic acid, ethanesulfonic acid, benzenesulfonic acid, p-toluenesulfonic acid, naphthalene sulfonic acid, acetic acid, trifluoroacetic acid, citric acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, etc.; amino acid salts formed with glycine, arginine, lysine, etc.; and amine salts formed with trimethylamine, triethylamine, ammonia, pyridine, picoline, etc. However, the present invention is not limited thereto.

In the present invention, “OO prodrug” refers to a substance which converts into its active form “OO” by the action of enzymes or chemicals in the body. For instance, a “candesartan prodrug” refers to a substance which is decomposed in vivo into an active ingredient candesartan.

The present invention provides a pharmaceutical formulation wherein a non-dihydropyridine calcium channel blocker is absorbed in the liver 2 to 4 hours after absorption of ARB.

Hereinafter, individual ingredients of the pharmaceutical formulation in accordance with the present invention will be described in more detail.

1. Prior-Release Compartment

The “prior-release compartment” refers to a compartment which is released ahead of the “delayed-release compartment” in the pharmaceutical formulation of the present invention. The prior-release compartment contains ARB and if any, an isomer thereof or a pharmaceutically acceptable salt thereof as a pharmacologically active ingredient, and may further contain pharmaceutically acceptable additives, if necessary.

In the present invention, the prior-release compartment can be prepared in the form of a mixture, granule, pellet or tablet, by subjecting a pharmacologically active ingredient together with pharmaceutically acceptable additives to conventional processes for the preparation of an oral formulation, such as mixing, kneading, drying and granulation. Further, when direct tableting is not feasible due to poor fluidity, the prior-release compartment may be granulated by pressing, granulation and sieving.

(1) Pharmacologically Active Ingredients

The prior-release compartment contains ARB, or if any, an isomer thereof or a pharmaceutically acceptable salt thereof as a pharmacologically active ingredient.

With regard to the formulation of the present invention, the content of ARB, which is a pharmacologically active ingredient, in the prior-release compartment, is in the range of 1 to 1000 mg/day, preferably 2.5 to 600 mg/day, based on the formulation (a total of 200 to 1200 mg), for an adult (adult male weighing 65 to 75 kg).

Specifically, when ARB is losartan, a content thereof in a unit formulation is preferably in the range of 12.5 to 100 mg, and 80 to 320 mg for valsartan, 20 to 80 mg for telmisartan, 400 to 600 mg for eprosartan, 75 to 300 mg for irbesartan, 4 to 32 mg for candesartan and 5 to 40 mg for olmesartan.

ARB in the prior-release compartment is released at a level of more than 60% of a total amount of ARB in a unit formulation within one hour after initiation of the release thereof, whereby drug efficacy can be rapidly exerted. For example, when ARB is losartan or telmisartan, ARB is released at a level of more than 80% of a total amount of ARB in the unit formulation within 30 minutes after initiation of the release thereof.

(2) Pharmaceutically Acceptable Additives

The prior-release compartment of the present invention may contain additives such as pharmaceutically acceptable diluent, binder, disintegrant, lubricant, pH-adjusting agent, stabilizer and solubilizer, within the range where effects of the present invention are not impaired.

In the prior-release compartment of the present invention, the content of the additive is in the range of 0.01 to 100 parts by weight, relative to 1 part by weight of ARB.

Examples of the diluent that can be used in the prior-release compartment of the present invention may include starch, microcrystalline cellulose, lactose (lactose hydrate), glucose, D-mannitol, alginate, an alkaline earth metal salt, clay, polyethylene glycol, anhydrous dibasic calcium phosphate, and a mixture thereof.

Examples of the binder that can be used in the prior-release compartment of the present invention may include starch, microcrystalline cellulose, highly dispersive silica, mannitol, sucrose, lactose hydrate, polyethylene glycol, polyvinylpyrrolidone (povidone), hydroxypropylmethylcellulose, hydroxypropylcellulose, natural gum, synthetic gum, copovidone, gelatin, and a mixture thereof.

Examples of the disintegrant that can be used in the prior-release compartment of the present invention may include starches or modified starches such as sodium starch glycolate, corn starch, potato starch, and pregelatinized starch; clays such as bentonite, montmorillonite, and veegum; celluloses such as microcrystalline cellulose, hydroxypropylcellulose, and carboxymethylcellulose; algins such as sodium alginate, and alginic acid; crosslinked celluloses such as croscarmellose, and croscarmellose sodium; gums such as guar gum, and xanthan gum; crosslinked polymers such as crosslinked polyvinylpyrrolidone (crospovidone); effervescent agents such as sodium bicarbonate and citric acid, and mixtures thereof.

Examples of the lubricant that can be used in the prior-release compartment of the present invention may include talc, stearic acid, magnesium stearate, calcium stearate, sodium lauryl sulfate, hydrogenated vegetable oil, sodium benzoate, sodium stearyl fumarate, glyceryl behenate, glyceryl monolaurate, glyceryl monostearate, glyceryl palmitostearate, and mixtures thereof.

Examples of the pH-adjusting agent that can be used in the prior-release compartment of the present invention may include acidulants such as acetic acid, adipic acid, ascorbic acid, malic acid, succinic acid, tartaric acid, fumaric acid, and citric acid, and basifying agents such as precipitated calcium carbonate, aqueous ammonia, and meglumine.

Examples of the stabilizer that can be used in the prior-release compartment of the present invention may include alkalizers such as alkali metal salts, alkaline earth metal salts, or mixtures thereof. Examples of the alkali metal salt and the alkaline earth metal salt include sodium hydroxide, calcium phosphate, calcium carbonate, sodium carbonate, sodium hydrogen carbonate, magnesium oxide, magnesium carbonate, sodium citrate, and tribasic calcium phosphate.

Examples of the solubilizer that can be used in the prior-release compartment of the present invention may include sodium lauryl sulfate, polyoxyethylene sorbitan fatty acid ester (such as polysorbate), and docusate sodium.

In addition, the formulation of the present invention may optionally contain pharmaceutically acceptable additives such as various additives selected from a colorant and a fragrance.

The range of the additive that can be used in prior-release compartment of the present invention is not limited to the above-mentioned additives, and the additive may be used in a conventional dose which can be suitably selected by those skilled in the art.

2. Delayed-Release Compartment

In the present invention, the “delayed-release compartment” refers to a compartment whose active ingredient is released at a certain time interval after the release of the active ingredient of the “prior-release compartment”. The delayed-release compartment contains (1) a pharmacologically active ingredient, i.e., a non-dihydropyridine calcium channel blocker, an isomer thereof or a pharmaceutically acceptable salt thereof and (2-1) a release-controlling material or (2-2) an osmo-regulator and a semi-permeable membrane coating base, and (3), if necessary, pharmaceutically acceptable additive(s).

(1) Pharmacologically Active Ingredients

The pharmacologically active ingredient of the delayed-release compartment is a non-dihydropyridine calcium channel blocker, or if any, an isomer thereof or a pharmaceutically acceptable salt thereof. As the active ingredient in the delayed-release compartment, the content of the non-dihydropyridine calcium channel blocker in a unit formulation is in the range of about 1 to 1000 mg/unit formulation, and preferably 2 to 500 mg.

Specifically, when the non-dihydropyridine calcium channel blocker is diltiazem, the content thereof in a unit formulation is preferably in the range of 120 to 420 mg, and 40 to 320 mg for verapamil, 50 to 100 mg for gallopamil, 25 to 50 mg for cinnarizine and 5 to 50 mg for flunarizine, respectively.

When the formulation of the present invention is orally administered, the non-dihydropyridine calcium channel blocker is released 2 hours after the release of ARB is initiated and is released at a level of less than 60% of a total amount of a non-dihydropyridine calcium channel blocker in a unit formulation up to 4 hours after the release of ARB is initiated.

(2-1) Release-Controlling Materials

The delayed-release compartment in the pharmaceutical formulation of the present invention contains at least one release-controlling material selected from the group consisting of an enteric polymer, a water-insoluble polymer, a hydrophobic compound and a hydrophilic polymer. The delayed-release compartment preferably contains at least one release-controlling material selected from a water-insoluble polymer and an enteric polymer.

In the delayed-release compartment of the present invention, the content of the release-controlling material is in the range of 0.05 to 100 parts by weight, relative to 1 part by weight of the non-dihydropyridine calcium channel blocker. If the content of the release-controlling material is lower than the above-specified range, it may be difficult to achieve a sufficient delayed-release property. On the other hand, if the content of the release-controlling material is higher than the above-specified range, significant clinical effects cannot be achieved due to delayed release of the drug.

The enteric polymer refers to a polymer which is insoluble or stable under the acidic conditions of less than pH 5, and is dissolved or degraded under the specific pH conditions of pH 5 or higher. The enteric polymer that can be used in the present invention is at least one selected from the group consisting of an enteric cellulose derivative, an enteric acrylic acid copolymer, an enteric maleic acid copolymer, and an enteric polyvinyl derivative. The enteric cellulose derivative is at least one selected from hydroxypropylmethylcellulose acetate succinate, hydroxypropylmethylcellulose phthalate, hydroxymethylethylcellulose phthalate, cellulose acetate phthalate, cellulose acetate succinate, cellulose acetate maleate, cellulose benzoate phthalate, cellulose propionate phthalate, methylcellulose phthalate, carboxymethylethylcellulose, ethylhydroxyethylcellulose phthalate, and methylhydroxyethylcellulose; the enteric acrylic acid copolymer is at least one selected from a styrene/acrylic acid copolymer, a methyl acrylate/acrylic acid copolymer, a methyl acrylate/methacrylic acid copolymer (e.g., Acryl-EZE), a butyl acrylate/styrene/acrylic acid copolymer, a methacrylic acid/methyl methacrylate copolymer (e.g., Eudragit L 100 or Eudragit S, Evonik, Germany), a methacrylic acid/ethyl acrylate copolymer (e.g., Eudragit L 100-55, Evonik, Germany), and a methyl acrylate/methacrylic acid/octyl acrylate copolymer; the enteric maleic acid copolymer is at least one selected from a vinylacetate/maleic anhydride copolymer, a styrene/maleic anhydride copolymer, a styrene/maleic monoester copolymer, a vinyl methyl ether/maleic anhydride copolymer, an ethylene/maleic anhydride copolymer, a vinyl butyl ether/maleic anhydride copolymer, an acrylonitrile/methyl crylate/maleic anhydride copolymer, and a butyl acrylate/styrene/maleic anhydride copolymer; and the enteric polyvinyl derivative is at least one selected from polyvinylalcohol phthalate, polyvinylacetal phthalate, polyvinylbutyrate phthalate, and polyvinylacetacetal phthalate. The enteric polymer in the formulation of the present invention is preferably at least one selected from hydroxypropylmethylcellulose phthalate and a methyl acrylate/methacrylic acid copolymer.

The content of the enteric polymer in accordance with the present invention may be in the range of 0.1 to 20 parts by weight, preferably 0.5 to 10 parts by weight, relative to the non-dihydropyridine calcium channel blocker. If the content of the enteric polymer is lower than 0.1 parts by weight, the enteric polymer may be easily dissolved at a pH of less than 5. On the other hand, if the content of the enteric polymer is higher than 20 parts by weight, this may lead to an unnecessary increase in a total weight of the formulation or excessively delayed release thereof.

The water-insoluble polymer refers to a pharmaceutically acceptable water-insoluble polymer which controls the release of a drug. The water-insoluble polymer that can be used in the present invention is at least one selected from the group consisting of polyvinyl acetate, a water-insoluble polymethacrylate copolymer {e.g. poly(ethyl acrylate, methyl methacrylate) copolymer, a poly(ethyl acrylate, methyl methacrylate, trimethylaminoethyl methacrylate) copolymer (e.g. Eudragit RS PO)}, ethylcellulose, cellulose ester, cellulose ether, cellulose acylate, cellulose diacylate, cellulose triacylate, cellulose acetate, cellulose diacetate, and cellulose triacetate. In the formulation of the present invention, the water-insoluble polymer is preferably ethylcellulose.

The content of the water-insoluble polymer in accordance with the present invention may be in the range of 0.1 to 30 parts by weight, preferably 0.5 to 20 parts by weight, relative to the non-dihydropyridine calcium channel blocker. If the content of the water-insoluble polymer is lower than 0.1 parts by weight, release of the drug may be not controlled. On the other hand, if the content of the water-insoluble polymer is higher than 30 parts by weight, release of the drug may be excessively delayed.

The hydrophobic compound refers to a pharmaceutically acceptable water-insoluble material which controls the release of a drug. The hydrophobic compound that can be used in the present invention is selected from the group consisting of fatty acid and fatty acid ester, fatty acid alcohol, wax, inorganic material, and a mixture thereof. The fatty acid or fatty acid ester is at least one selected from glyceryl palmitostearate, glyceryl stearate, glyceryl behenate, cetyl palmitate, glyceryl monooleate and stearic acid; the fatty acid alcohol is at least one selected from cetostearyl alcohol, cetyl alcohol and stearyl alcohol; the wax is at least one selected from carnauba wax, beeswax and microcrystalline wax; and the inorganic material is at least one selected from talc, precipitated calcium carbonate, calcium hydrogen phosphate, zinc oxide, titanium oxide, kaolin, bentonite, montmorillonite and veegum.

The content of the hydrophobic compound in accordance with the present invention may be in the range of 0.1 to 20 parts by weight, preferably 0.5 to 10 parts by weight, relative to the non-dihydropyridine calcium channel blocker. If the content of the hydrophobic compound is lower than 0.1 parts by weight, release of the drug may be not controlled. On the other hand, if the content of the hydrophobic compound is higher than 20 parts by weight, release of the drug may be excessively delayed.

The hydrophilic polymer refers to a pharmaceutically acceptable water-soluble polymer which controls the release of a drug. The hydrophilic polymer that can be used in the present invention is selected from the group consisting of a saccharide, a cellulose derivative, gum, a protein, a polyvinyl derivative, a hydrophilic polymethacrylate copolymer, a polyethylene derivative, a carboxyvinyl copolymer and a mixture thereof. The saccharide is at least one selected from dextrin, polydextrin, dextran, pectin and a pectin derivative, alginate, polygalacturonic acid, xylan, arabinoxylan, arabinogalactan, starch, hydroxypropyl starch, amylase and amylopectin; the cellulose derivative is at least one selected from hydroxypropylmethylcellulose, hydroxypropylcellulose, hydroxymethylcellulose, hydroxyethylcellulose, methylcellulose, sodium carboxymethylcellulose and hydroxyethylmethylcellulose; the gum is at least one selected from guar gum, locust bean gum, tragacanth, carrageenan, gum acacia, gum arabic, gellan gum and xanthan gum; the protein is at least one selected from gelatin, casein and zein; the polyvinyl derivative is at least one selected from polyvinyl alcohol, polyvinyl pyrrolidone and polyvinylacetal diethylaminoacetate; the hydrophilic polymethacrylate copolymer is at least one selected from a poly(butyl methacrylate, (2-dimethylaminoethyl)methacrylate, methyl methacrylate) copolymer, a poly(methacrylate, methyl methacrylate) copolymer and a poly(methacrylate, ethyl acrylate) copolymer; the polyethylene derivative is at least one selected from polyethylene glycol and polyethylene oxide; and the carboxyvinyl polymer is carbomer. The hydrophilic polymer in the formulation of the present invention is preferably carbomer.

The content of the hydrophilic polymer in accordance with the present invention may be in the range of 0.05 to 30 parts by weight, preferably 0.5 to 20 parts by weight, relative to 1 part by weight of the non-dihydropyridine calcium channel blocker. If the content of the hydrophilic polymer is lower than 0.05 parts by weight, release of the drug may be not controlled. On the other hand, if the content of the hydrophilic polymer is higher than 30 parts by weight, release of the drug may be excessively delayed.

(2-2) Osmo-Regulator and Semi-Permeable Membrane Coating Base

The delayed-release compartment of the present invention may be a compartment which contains an osmo-regulator and is coated by a semi-permeable membrane coating base.

In the delayed-release compartment of the present invention, the osmo-regulator is at least one selected from the group consisting of magnesium sulfate, magnesium chloride, sodium chloride, lithium chloride, potassium sulfate, sodium sulfate, lithium sulfate and a mixture thereof. Preferred is sodium chloride or sodium sulfate.

The content of the osmo-regulator may be in the range of 0.05 to 30 parts by weight, preferably 0.1 to 20 parts by weight, relative to 1 part by weight of the non-dihydropyridine calcium channel blocker. If the content of the osmo-regulator is lower than 0.1 parts by weight, osmotic pressure-generating effects are insignificant. On the other hand, if the content of the osmo-regulator is higher than 30 parts by weight, this may lead to an unnecessary increase in a total weight of the formulation or a difficulty in achieving an optimum release rate of the drug.

In the delayed-release compartment of the present invention, the semi-permeable membrane coating base is a pharmaceutically acceptable base and refers to a material that is blended in a coating layer of the pharmaceutical formulation to form a membrane through which some ingredients can pass but other ingredients cannot pass. The above-mentioned water-insoluble polymers may also be used as the semi-permeable coating base. For example, the semi-permeable membrane coating base that can be used in the present invention is at least one selected from the group consisting of polyvinyl acetate, a polymethacrylate copolymer, ethylcellulose, cellulose ester, cellulose ether, cellulose acylate, cellulose diacylate, cellulose triacylate, cellulose acetate, cellulose diacetate, cellulose triacetate and a mixture thereof.

The content of the semi-permeable membrane coating base may be in the range of 0.05 to 30 parts by weight, preferably 0.1 to 20 parts by weight, relative to 1 part by weight of the non-dihydropyridine calcium channel blocker. If the content of the semi-permeable membrane coating base is lower than 0.05 parts by weight, it may be difficult to achieve a sufficient time-lag. On the other hand, if the content of the semi-permeable membrane coating base is higher than 30 parts by weight, there is a problem associated with no release of the drug or an excessively long time-lag of 9 hours.

(3) Pharmaceutically Acceptable Additives

In addition to (2-1) a release-controlling material and (2-2) an osmo-regulator and a semi-permeable membrane coating base, the formulation of the present invention may further contain commonly used additives such as pharmaceutically acceptable diluent, binder, disintegrant, lubricant, pH-adjusting agent, anti-foaming agent, and solubilizer, within the range where the effects of the present invention are not impaired and within the range where delayed-release properties are not compromised.

Examples of the diluent that can be used in the present invention may include starch, microcrystalline cellulose, lactose hydrate, glucose, mannitol, D-mannitol, alginate, an alkaline earth metal salt, clay, polyethylene glycol, anhydrous dibasic calcium phosphate, and a mixture thereof. Examples of the binder that can be used in the present invention include starch, microcrystalline cellulose, highly dispersive silica, mannitol, sucrose, lactose hydrate, polyethylene glycol, polyvinylpyrrolidone, hydroxypropylmethylcellulose, hydroxypropylcellulose, natural gum, synthetic gum, copovidone, povidone, gelatin, and a mixture thereof.

Examples of the disintegrant that can be used in the present invention may include starches or modified starches such as sodium starch glycolate, corn starch, potato starch, and pregelatinized starch, clays such as bentonite, montmorillonite, and veegum, celluloses such as microcrystalline cellulose, hydroxypropylcellulose, and carboxymethylcellulose, algins such as sodium alginate, and alginic acid, crosslinked celluloses such as croscarmellose sodium, gums such as guar gum, and xanthan gum, crosslinked polymers such as crosslinked polyvinylpyrrolidone (crospovidone), effervescent agents such as sodium bicarbonate and citric acid, and mixtures thereof.

Examples of the lubricant that can be used in the present invention may include talc, stearic acid, magnesium stearate, calcium stearate, sodium lauryl sulfate, hydrogenated vegetable oil, sodium benzoate, colloidal silicon dioxide, sodium stearyl fumarate, glyceryl behenate, glyceryl monolaurate, glyceryl monostearate, glyceryl palmitostearate, polyethylene glycol and aluminum magnesium metasilicate.

As the pharmaceutically acceptable additive, examples of the pH-adjusting agent that can be used in the present invention may include acidulants such as acetic acid, adipic acid, ascorbic acid, malic acid, succinic acid, tartaric acid, fumaric acid, and citric acid, and basifying agents such as precipitated calcium carbonate, aqueous ammonia, and meglumine.

As the pharmaceutically acceptable additive of the present invention, examples of the anti-foaming agent may include dimethicone, oleyl alcohol, propylene glycol alginate, and simethicone (such as simethicone emulsion).

As the pharmaceutically acceptable additive of the present invention, examples of the solubilizer may include sodium lauryl sulfate, polyoxyethylene sorbitan fatty acid ester (such as polysorbate), and docusate sodium. Further, a plasticizer, such as Myvacet, triethyl citrate or polyethylene glycol, may also be added.

In addition, the formulation of the present invention may contain pharmaceutically acceptable additives such as various additives selected from a colorant and a fragrance. The range of the additives that can be used in the present invention is not limited to the above-mentioned additives, and the additive may be used in a conventional dose which can be suitably selected by those skilled in the art.

Further, in the delayed-release formulation of the present invention, as a binding solvent and a solvent for the delayed-release additive, there may be employed purified water, ethanol, methylene chloride, and the like. More preferred are purified water and ethanol.

In the pharmaceutically acceptable additive of the present invention, the range of the usable additive is not limited to the above-mentioned additive, and the additive may be used in a conventional dose which can be suitably selected by those skilled in the art.

The formulation of the present invention can be prepared into various formulations, for example, tablets (such as uncoated tablets, coated tablets, multi-layered tablets, and press-coated tablets), powders, granules, or capsules.

The formulation of the present invention may be in the form of an uncoated tablet which is obtained by optionally subjecting prior-release compartment-forming granules or the like and delayed-release compartment-forming granules or the like to post-mixing with additive(s), followed by compression, such that the prior-release compartment and the delayed-release compartment are present within a single tablet whereby active ingredients of individual compartments are separately released to exhibit the efficacy of each drug.

The formulation of the present invention may be in the form of a two-phase matrix tablet including a delayed-release compartment and a prior-release compartment enclosing the delayed-release compartment.

Further, the formulation of the present invention may be in the form of a film-coated tablet including a tablet of a delayed-release compartment and a film-coating layer of a prior-release compartment enclosing the exterior of the tablet, whereby irbesartan of the film-coating layer is first released as the film-coating layer is dissolved.

Further, the formulation of the present invention may be in the form of a multi-layered tablet having a multi-layered structure of a delayed-release compartment and a prior-release compartment, each compartment of which is obtained by mixing the granules constituting the delayed-release compartment and the prior-release compartment with pharmaceutical additives, and compressing the mixture into a double-layered or triple-layered tablet, using a multiple tablet press. Individual layers constituting the multi-layered tablet may be parallel to each other. The resulting formulation is a tablet for oral administration which was formulated to achieve the prior-release and delayed-release of drugs according to individual layers.

Further, the formulation of the present invention may be in the form of a press-coated tablet including an inner core of a delayed-release compartment and an outer layer of a prior-release compartment enclosing the outer surface of the inner core. The press-coated tablet may be an osmotic press-coated tablet. The osmotic press-coated tablet is a formulation wherein the tablet mix is compressed into a tablet in a manner that an osmo-regulator is incorporated for the delayed-release of a drug, the tablet surface is coated with an osmotic semi-permeable membrane coating base to prepare an inner core, a granule constituting the prior-release compartment is mixed with pharmaceutical additives to prepare an outer layer, followed by compression to form a formulation having a delayed-release inner core and a prior-release layer enclosing the surface of the inner core.

The formulation of the present invention may be in the form of a capsule containing a particle, granule, pellet, or tablet of a delayed-release compartment and a particle, granule, pellet, or tablet of a prior-release compartment.

The tablet formed of the delayed-release compartment of the capsule may be an osmotic coated tablet which contains an osmo-regulator inside the tablet and has a semi-permeable membrane coating base on the surface of the tablet.

The material for the capsule may be one selected from gelatin, succinate gelatin, hydroxypropylmethylcellulose, and a mixture thereof.

The formulation of the present invention may further include a coating layer on the outside of the delayed-release compartment and/or the prior-release compartment. That is, the surface of particles, granules, pellets, or tablets formed of the delayed-release compartment and/or the prior-release compartment may be coated for the purpose of delayed release of drugs or stability of the formulation.

The formulation of the present invention may be a formulation in the form of an uncoated tablet without further coating, or otherwise, if necessary, may be provided in the form of a coated tablet further including a coating layer formed on the outside of the formulation. The formation of a coating layer can provide a formulation which is capable of further securing stability of pharmacologically active ingredients.

A method for forming the coating layer may be suitably selected by a skilled person in the art, from among methods capable of forming a film-like coating layer on the surface of the tablet layer, such as a fluidized-bed coating method and a pan coating method. Preferably, a pan coating method may be used.

The coating layer may be formed by using a film-forming agent, a film-forming aid or a mixture thereof. For example, the film-forming agent may be cellulose derivatives such as hydroxypropylmethylcellulose and hydroxypropylcellulose, saccharide derivatives, polyvinyl derivatives, waxes, fats, gelatin and mixtures thereof, and the film-forming aid may be polyethylene glycol, ethylcellulose, glyceride, titanium oxide, talc, diethyl phthalate and mixtures thereof.

The content of the coating layer may be in the range of 0.5 to 15% by weight based on the total weight of the tablet.

The pharmaceutical formulation of the present invention can be formulated into a desired dosage form depending on individual diseases or ingredients, by an appropriate method known in the art, for example, using the principle of the chronotherapy as disclosed in Chronotherapeutics (2003, Peter Redfern, PhP), specifically by a method including the following steps.

Step 1 is a step of obtaining a delayed-release granule or tablet by subjecting a non-dihydropyridine calcium channel blocker and one or two release-controlling materials selected from the group consisting of an enteric polymer, a water-insoluble polymer, a hydrophobic compound, and a hydrophilic polymer together with a pharmaceutically acceptable conventional additive to mixing, kneading, drying, granulation or coating, and compression, or of obtaining a delayed-release granule or tablet by subjecting a non-dihydropyridine calcium channel blocker and an osmo-regulator together with a conventional pharmaceutically acceptable additive to mixing, kneading, drying, granulation or compression, followed by coating with a semi-permeable membrane coating base.

Step 2 is a step of obtaining a prior-release granule or tablet by subjecting ARB together with a conventional pharmaceutically acceptable additive to conventional processes for producing oral solid formulations, for example, mixing, kneading, drying, granulation or coating, and compression.

Step 3 is a step of obtaining a formulation for oral administration by mixing the granule or tablet obtained in each of Steps 1 and 2 with a pharmaceutically acceptable excipient and either compressing the mixture into a tablet or filling the mixture in a capsule for oral administration.

Step 1 may be carried out after Step 2, or Step 1 may be carried out simultaneously with Step 2.

The combination formulation of the present invention can be prepared according to the above procedure, and a formulation method will be described in more detail hereinafter, but the present invention is not limited thereto.

1. Preparation of Two-Phase Matrix Tablets

The particles or granules prepared in Step 1 are optionally coated with a release-controlling material and then mixed with the granules prepared in Step 2, followed by compression into uniform weight, thereby preparing tablets. The resulting tablets may be film-coated for the purpose of improving the stability or shape, if necessary.

2. Preparation of Film-Coated Tablets Containing Pharmacologically Active Ingredients

The coated tablets or granules prepared in Step 1 are optionally coated with a release-controlling material and dried, followed by compression into uniform weight and optionally further coating to prepare tablets. In addition, ARB is dissolved and dispersed in a water-soluble film coating solution and is coated on the outer layer of the tablets obtained in Step 1 to thereby prepare oral film-coated tablets containing pharmacologically active ingredients in the film coating.

3. Preparation of Multi-Layered Tablets

The granules prepared in Step 1 are optionally coated with a release-controlling material, and dried. The dried granules are compressed with the granules prepared in Step 2 by using a multi-layered tablet press, thereby obtaining a double-layered tablet. According to the formulation design or if necessary, a triple or more multi-layered tablet may also be prepared by further adding a release adjuvant layer on the double-layered tablet. A coated multi-layered tablet may be prepared by coating the multi-layered tablet.

4. Preparation of Press-Coated Tablets

The coated tablets or granules prepared in Step 1 are optionally coated with a release-controlling material and dried, followed by compression into uniform weight. The resulting granules are used as an inner core optionally after performing further coating, and compressed with the granules prepared in Step 2 by using a press-coated tablet press, thereby providing press-coated tablets where the surface of the tablet of Step 1 is enclosed by the prior-release layer. Coated press-coated tablets may be prepared by coating the press-coated tablets.

5. Preparation of Capsules (Containing Granules or Tablets)

The granules prepared in Step 1 are optionally coated with a release-controlling material, and dried. The dried granules together with the granules prepared in Step 2 may be placed in a capsule filling machine, and filled in a capsule having a given size at an effective amount of each main ingredient, thereby preparing a capsule.

6. Preparation of Capsules (Pellets)

(1) A non-dihydropyridine calcium channel blocker, a release-controlling material, and if necessary, pharmaceutically acceptable additives are dissolved or suspended in water, an organic solvent, or a mixed solvent. This solution or suspension is coated on sugar spheres and dried, and if necessary, coated with one or more release-controlling materials dissolved in water, an organic solvent, or a mixed solvent, followed by drying. The mixture may be mixed with the granules prepared in Step 2 or the tablets obtained in Step 3 and then filled in capsules using a capsule filling machine, thereby preparing capsules.

(2) An ARB and pharmaceutically acceptable additives may be dissolved or suspended in water, an organic solvent or a mixed solvent, coated on sugar spheres, followed by drying, and mixed with the non-dihydropyridine calcium channel blocker-containing controlled-release pellets of Section (1) and filled in capsules using a capsule filling machine to prepare capsules.

7. Preparation of Kit

The non-dihydropyridine calcium channel blocker-containing formulation of Step 1 and the ARB-containing formulation of Step 2 can be filled in a foil, blister, or bottle to prepare a kit for concurrent administration of different drugs.

The above-stated pharmaceutical formulation of the present invention is a chronotherapeutic formulation containing a non-dihydropyridine calcium channel blocker and ARB as pharmacologically active ingredients, which is configured to achieve administration once a day in the evening (between 17:00 and 23:00), thereby providing convenient education of medication instruction as compared to concurrent administration of separate formulations of individual active ingredients, is capable of reducing antagonism-induced side effects due to no occurrence of antagonism between drugs, and is also capable of providing improved effects of individual drugs in control of blood pressure and lipids, as compared to effects exhibited when these drugs are separately administered.

Specific effects of the pharmaceutical formulation in accordance with the present invention are as follows.

1) Maximized Efficacy of Two Drugs

The risk of hypertension is highest in the morning, due to a sudden rise of blood pressure upon getting-up in the morning [William et al., The American Journal of Cardiology. vol. 100(3) (2007) p s10-s16].

However, the pharmaceutical formulation for evening administration in accordance with the present invention maintains constant blood pressure for 24 hours and prevents complications by configuring such that ARB is prior-released to effectively lower blood pressure up to the dawn, and the non-dihydropyridine calcium channel blocker is released after a certain lag time of release, that is, 2 hours after administration of the drug whereby blood pressure after the dawn is effectively dropped.

2) Patients Compliance and Medication Convenience

The pharmaceutical formulation of the present invention not only achieves the optimization of a drug delivery time by allowing to release individual drugs at a certain rate based on the principle of Chronotherapy where administration of different drugs is made to act with different time of pharmacological activities in vivo, but also has high patents' compliance due to increased medication convenience by the administration once a day.

3) Minimization of Side Effects Due to Drug-Drug Interaction

When different drugs are absorbed in the body at the same time, drug-drug interaction occurs and has effects on absorption, metabolism, distribution and excretion of drugs due to the nature of individual drugs [Michael et al., Current Problems in Cardiology. vol. 33 (12) (2008). p 703-768].

The chronotherapeutic pharmaceutical formulation of the present invention can avoid the drug-drug interaction by controlling the release of drugs such that the non-dihydropyridine calcium channel blocker is released after the release of ARB whereby the non-dihydropyridine calcium channel blocker is absorbed 2 to 4 hours after ARB is prior-released and sufficiently absorbed in the liver, thus resulting in no effects of the non-dihydropyridine calcium channel blocker on the metabolism of ARB. In this manner, drug interaction and side effects exhibited by simple combination formulations are reduced.

Further, all kinds of drugs exhibit therapeutic effects and side effects which are contradictory to each other. That is, the presence of drug in blood plays an importance role in exerting therapeutic effects and also serves to cause side effects. Therefore, when the blood level of the drug excessively rises within a short period of time, this may lead to an increase in the risk of side effects. That is, when two different drugs reach the maximum blood level (Cₘₐₓ) at the same time, this may lead to the risk of unexpected side effects because an excessive amount of the drugs distributes in blood within a short period of time.

However, in the pharmaceutical formulation of the present invention, the non-dihydropyridine calcium channel blocker of the delayed-release compartment is released after a release lag time of 2 to 4 hours and reaches its Tₘₐₓ after Tₘₐₓ of the prior-released ARB has passed, so that appearance of Cₘₐₓ of each drug does not overlap within a short period of time, whereby side effects due to interactions between two drugs can be minimized.

Accordingly, the present invention provides a pharmaceutical formulation which contains the pharmaceutical formulation of the present invention and is intended for administration between 5:00 p.m. to 11:00 p.m.

A dose of the inventive formulation is appropriately determined depending on absorptivity of active ingredients in vivo, inactivation rate and excretion rate, age, sex and conditions of patients, and the like. For adults, the daily dose of formulation is usually in the range of 2 to 2000 mg, preferably 4 to 1100 mg in terms of a total of the non-dihydropyridine calcium channel blocker and ARB, such that anti-hypertensive effects, lipid-lowering effects and complication-preventing effects can be exhibited.

Further, the present invention provides a method for treating a cardiovascular disease, including administering the pharmaceutical formulation of the present invention to a mammal. Preferably, provided is a method for treating hypertension and hyperlipidemia or related cardiovascular diseases or metabolic syndromes, including administering the pharmaceutical formulation of the present invention to a mammal, once a day, between 5:00 p.m. to 11:00 p.m.

Cardiovascular disease is a very broad disease generically covering all of cardiovascular diseases and other blood vessel diseases including cerebrovascular diseases. Heart disease is represented by ischemic heart diseases (myocardial infarction, angina pectoris, etc.) due to the progression of arteriosclerosis and includes hypertension, heart failure, arrhythmia, myocardiopathy, endocarditis, etc. Examples of blood vessel diseases include cerebral stroke (palsy), peripheral vascular diseases, etc. Further, cardiovascular disease includes hypertension and complications thereof of people suffering from a metabolic syndrome with combined manifestation of hypertension, diabetes, obesity, hyperlipidemia, and coronary artery diseases.

### Advantageous Effects

The pharmaceutical formulation of the present invention is a pharmaceutical formulation containing a non-dihydropyridine calcium channel blocker and ARB, which is designed based on Xenobiotics and Chronotherapy, and exhibits reduction of side effects due to co-administration of single drugs and avoidance of drug-drug interaction, in conjunction with high therapeutic effectiveness in treatment of hypertension and hyperlipidemia and prevention of complications in people with metabolic syndromes and improved patents' compliance and optimization of a drug delivery time.

### DESCRIPTION OF THE DRAWINGS

FIG. 1 is a graph showing the dissolution profiles of a losartan single drug and a diltiazem immediate-release tablet single drug, and losartan and diltiazem in a pharmaceutical formulation of Example 2, according to Experimental Example.

FIG. 2 is a graph showing the dissolution profiles of a losartan single drug and a diltiazem sustained-release tablet single drug, and losartan and diltiazem in a pharmaceutical formulation of Example 5, according to Experimental Example.

FIG. 3 is a graph showing the dissolution profiles of a valsartan single drug and a diltiazem sustained-release tablet single drug, and valsartan and diltiazem in a pharmaceutical formulation of Example 7, according to Experimental Example.

FIG. 4 is a graph showing the dissolution profiles of a telmisartan single drug and a diltiazem sustained-release tablet single drug, and telmisartan and diltiazem in a pharmaceutical formulation of Example 8, according to Experimental Example.

FIG. 5 is a graph showing the dissolution profiles of losartan and diltiazem in a pharmaceutical formulation of Example 16, and losartan and verapamil in a pharmaceutical formulation of Example 19, according to Experimental Example.

FIG. 6 is a graph showing the dissolution profiles of losartan and diltiazem in pharmaceutical formulations of Examples 23 and 26, according to Experimental Example.

### MODE FOR INVENTION

Now, the present invention will be described in more detail with reference to the following Examples. These examples are provided only for illustrating the present invention and should not be construed as limiting the scope and spirit of the present invention.

### Example 1

### Preparation of Diltiazem-Losartan Single Tablets

(1) Preparation of Losartan Potassium Granules

According to the ingredients and contents shown in Table 2 below, losartan potassium (Losartan K, Cipla), lactose (Lactose DCL-15, DMV), microcrystalline cellulose (Vivapur 102, JRS), pregelatinized starch (Starch 1500, Colorcon), and hydroxypropylcellulose (Klucel EXF, Aqualon) were weighed and sieved through a No. 20 sieve and then mixed in a double cone mixer for 15 minutes to prepare a mixture. After completion of the mixing process, sodium starch glycolate (Explotab, JRS) was added thereto, followed by further mixing for 8 minutes. Magnesium stearate (N of) was sieved through a No. 35 sieve and added thereto, followed by final mixing for 4 minutes to prepare losartan prior-release granules.

(2) Diltiazem Delayed-Release Granules

According to the ingredients and contents shown in Table 2 below, diltiazem hydrochloride (Diltiazem HCl, Ranbaxy) and microcrystalline cellulose (Vivapur101, JRS) were sieved through a No. 35 sieve and mixed in a double cone mixer for 5 minutes to prepare a mixture. Meanwhile, hydroxypropylcellulose (HPC-L, Hercules) was dissolved in purified water to prepare a binding solution. The mixture was placed in a fluidized bed granulator and kneaded with the binding solution to prepare granules, followed by drying. Meanwhile, ethylcellulose (Ethocel, Colorcon) and triethyl citrate (Duksan) were dissolved in an ethanol-methylene chloride mixture to prepare a film coating solution. The dried material was coated with the ethylcellulose film coating solution in a fluidized bed coater. After completion of the coating process, magnesium stearate was added thereto, followed by mixing for 4 minutes to prepare diltiazem delayed-release granules.

(3) Compression and Coating

The prepared diltiazem and losartan granules were mixed and compressed into tablets using a rotary tablet press equipped with a punch having a diameter of 10.0 mm (MRC-33: Sejong Machinery Co., Ltd., South Korea). Hydroxypropylmethylcellulose 2910 (Methocel, Colorcon), polyethylene glycol 6,000 (PEG600, Daejung) and titanium oxide (TiO₂, Hwawon) were dissolved in ethanol and methylene chloride to prepare a coating solution which was then coated on the compressed tablets under the conventional tablet coating conditions.

### Example 2

### Diltiazem-Losartan Single Tablets

According to the ingredients and contents shown in Table 2 below, the title single tablets were prepared in the same manner as in Example 1, except that upon preparation of diltiazem delayed-release granules, tablet granules were coated with ethylcellulose as a film coating base, followed by further coating with hydroxypropylmethylcellulose phthalate (HPMCP-55, Shin-Etsu) dissolved in an ethanol-methylene chloride mixture.

### Example 3

### Diltiazem-Losartan Film-Coated Tablets

(1) Preparation of Diltiazem Film-Coated Tablets

According to the ingredients and contents shown in Table 2 below, diltiazem hydrochloride and microcrystalline cellulose were sieved through a No. 35 sieve and mixed in a double cone mixer for 20 minutes. Meanwhile, hydroxypropylcellulose was dissolved in purified water to prepare a binding solution. The mixture was placed in a high-speed mixer and the binding solution was added thereto, followed by kneading to prepare granules. The prepared granules were dried in a hot-water dryer at 60° C. and sieved through a grinder equipped with a No. 25 sieve. The sieved material was placed in a double cone mixer and Carbomer 941 (Lubrizol) was added thereto, followed by mixing for 10 minutes. After completion of the mixing process, magnesium stearate was sieved through a No. 35 sieve and added to the mixture, followed by final mixing for 4 minutes to prepare diltiazem delayed-release granules. The granules were compressed into tablets using a rotary tablet press (MRC-33: Sejong Machinery Co., Ltd., South Korea), thereby preparing diltiazem tablets.

Thereafter, according to the ingredients and contents shown in Table 2 below, hydroxypropylmethylcellulose phthalate and Myvacet (Hwawon) were dissolved in an ethanolmethylene chloride mixture to prepare a film coating solution. The above-prepared diltiazem tablets were placed in a Hi-coater (SFC-30F, Sejong Machinery Co., Ltd., South Korea) and coated with the film coating solution.

(2) Preparation of Losartan-Containing Drug Coating Solution

According to the ingredients and contents shown in Table 2 below, losartan potassium, lactose, hydroxypropylmethylcellulose, and talc (Hwawon) were dissolved or dispersed in an ethanol-methylene chloride mixture to prepare a drug coating solution.

(3) Preparation of Film-Coated Tablets

The above-prepared diltiazem film-coated tablets were placed in a Hi-coater (SFC-30F, Sejong Machinery Co., Ltd., South Korea) and further coated with the losartan-containing drug coating solution. After completion of the coating process, further coating was carried out by a solution of hydroxypropylmethylcellulose 2910 and polyethylene glycol 6,000 in an ethanolmethylene chloride mixture.

### Example 4

### Diltiazem-Losartan Double-Layered Tablets

According to the ingredients and contents shown in Table 2 below, diltiazem delayed-release granules and losartan prior-release granules were prepared in the same manner as in Example 2. Both granules were placed in corresponding inlets of a multi-layered tablet press (MRC-37T: Sejong Machinery Co., Ltd., South Korea), followed by compression according to given contents of individual drugs. Hydroxypropylmethylcellulose 2910, polyethylene glycol 6,000 and titanium oxide were dissolved in an ethanol-methylene chloride mixture to prepare a coating solution which was then coated on the compressed tablets under the conventional tablet coating conditions.

### Example 5

### Diltiazem-Losartan Double-Layered Tablets

(1) Losartan Prior-Release Granules

According to the ingredients and contents shown in Table 2 below, losartan prior-release granules were prepared in the same manner as in preparation of losartan prior-release granules in Example 1.

(2) Diltiazem Delayed-Release Granules

According to the ingredients and contents shown in Table 2 below, diltiazem hydrochloride and microcrystalline cellulose were sieved through a No. 35 sieve and mixed in a double cone mixer for 20 minutes. Meanwhile, hydroxypropylcellulose was dissolved in purified water to prepare a binding solution. The mixture was placed in a high-speed mixer and the binding solution was added thereto, followed by kneading to prepare granules. The prepared granules were dried in a hot-water dryer at 60° C. and sieved through a grinder equipped with a No. 20 sieve. Meanwhile, hydroxypropylmethylcellulose phthalate was dissolved in an ethanol-methylene chloride mixture to prepare a solution which was then coated on the sieved material in a fluidized bed coater. The coated material was placed in a double cone mixer and Carbomer 941 was added thereto, followed by mixing for 10 minutes. After completion of the mixing process, magnesium stearate was sieved through a No. 35 sieve and added to the mixture, followed by final mixing for 4 minutes to prepare diltiazem delayed-release granules.

(3) Compression and Coating

The prepared diltiazem and losartan granules were placed in corresponding inlets of a multi-layered tablet press (MRC-37T: Sejong Machinery Co., Ltd., South Korea), followed by compression according to given contents of individual drugs, thereby preparing double-layered tablets. Meanwhile, hydroxypropylmethylcellulose 2910, polyethylene glycol 6,000 and titanium oxide were dissolved in an ethanol-methylene chloride mixture to prepare a coating solution which was then coated on the compressed tablets under the conventional tablet coating conditions.

### Example 6

### Diltiazem-Losartan Double-Layered Tablets

According to the ingredients and contents shown in Table 2 below, diltiazem-losartan double-layered tablets were prepared in the same manner as in Example 5, except that weight of the diltiazem layer and the losartan layer were increased.

### Example 7

### Diltiazem-Valsartan Double-Layered Tablets

According to the ingredients and contents shown in Table 2 below, diltiazem-valsartan double-layered tablets were prepared in the same manner as in Example 5, except that valsartan (Ranbaxy) was used in place of losartan potassium, and calcium phosphate (DCP A, Rhodia) was used in place of lactose.

### Example 8

### Diltiazem-Telmisartan Double-Layered Tablets

According to the ingredients and contents shown in Table 2 below, diltiazem-telmisartan double-layered tablets were prepared in the same manner as in Example 5, except that telmisartan (Cipla) was used in place of losartan potassium, and sodium hydroxide (NaOH, Daejung) was used in place of lactose.

### Example 9

### Diltiazem-Candesartan Double-Layered Tablets

According to the ingredients and contents shown in Table 2 below, diltiazem-candesartan double-layered tablets were prepared in the same manner as in Example 5, except that candesartan cilexetil (Ranbaxy) was used in place of losartan potassium.

### Example 10

### Diltiazem-Irbesartan Double-Layered Tablets

According to the ingredients and contents shown in Table 2 below, diltiazem-irbesartan double-layered tablets were prepared in the same manner as in Example 5, except that irbesartan (Ranbaxy) was used in place of losartan potassium.

### Example 11

### Diltiazem-Olmesartan Double-Layered Tablets

According to the ingredients and contents shown in Table 3 below, diltiazem-olmesartan double-layered tablets were prepared in the same manner as in Example 5, except that olmesartan medoxomil (Cipla) was used in place of losartan.

### Example 12

### Diltiazem-Eprosartan Double-Layered Tablets

According to the ingredients and contents shown in Table 3 below, diltiazem-eprosartan double-layered tablets were prepared in the same manner as in Example 5, except that eprosartan mesylate (Biocon) was used in place of losartan.

### Example 13

### Verapamil-Losartan Double-Layered Tablets

According to the ingredients and contents shown in Table 3 below, verapamil-losartan double-layered tablets were prepared in the same manner as in Example 5, except that verapamil hydrochloride (Verapamil HCl, Ranbaxy) and fumaric acid (Daejung) were used in place of diltiazem hydrochloride.

### Example 14

### Diltiazem-Losartan Triple-Layered Tablets

According to the ingredients and contents shown in Table 3 below, diltiazem-losartan triple-layered tablets were prepared in the same manner as in Example 5, except that tablet compression was performed such that a losartan prior-release layer was disposed on a first layer, a placebo layer (microcrystalline cellulose 150.0 mg, magnesium stearate 5.0 mg) was disposed on a second layer, and a diltiazem delayed-release layer was disposed on a third layer.

### Example 15

### Diltiazem-Losartan Inner Core Tablets

(1) Preparation of Losartan Potassium Granules

According to the ingredients and contents shown in Table 3 below, losartan potassium, lactose, microcrystalline cellulose, pregelatinized starch, and hydroxypropylcellulose were weighed and sieved through a No. 20 sieve and then mixed in a double cone mixer for 15 minutes to prepare a mixture. After completion of the mixing process, sodium starch glycolate was added thereto, followed by further mixing for 8 minutes. Magnesium stearate was sieved through a No. 35 sieve and added to the mixture, followed by final mixing for 4 minutes to prepare losartan prior-release granules.

(2) Diltiazem Delayed-Release Inner Core Tablets

According to the ingredients and contents shown in Table 3 below, diltiazem hydrochloride and microcrystalline cellulose were sieved through a No. 35 sieve and mixed in a double cone mixer for 20 minutes. Meanwhile, hydroxypropylcellulose was dissolved in purified water to prepare a binding solution. The mixture was placed in a high-speed mixer and the binding solution was added thereto, followed by kneading to prepare granules. The prepared granules were dried in a hot-water dryer at 60° C. and sieved through a grinder equipped with a No. 25 sieve. The sieved material was placed in a double cone mixer and Carbomer 941 was added thereto, followed by mixing for 10 minutes. After completion of the mixing process, magnesium stearate was sieved through a No. 35 sieve and added to the mixture, followed by final mixing for 4 minutes to prepare diltiazem delayed-release granules. The granules were compressed into tablets using a rotary tablet press (MRC-33: Sejong Machinery Co., Ltd., South Korea), thereby preparing diltiazem delayed-release inner core tablets.

(3) Compression and Coating

Using a press-coated tablet press (RUD-1: Kilian), press-coated tablets were prepared including the diltiazem core tablet as an inner core and the losartan granule-containing composition as an outer layer. Meanwhile, hydroxypropylmethylcellulose 2910, polyethylene glycol 6,000 and titanium oxide were dissolved in an ethanol-methylene chloride mixture to prepare a coating solution. The compressed press-coated tablets were coated with the coating solution in a Hi-coater (SFC-30N, Sejong Machinery Co., Ltd., South Korea) to prepare press-coated tablets.

### Example 16

### Diltiazem-Losartan Press-Coated Tablets

According to the ingredients and contents shown in Table 3 below, diltiazem-losartan press-coated tablets were prepared in the same manner as in Example 15, except that the diltiazem delayed-release inner core tablets were further coated with an enteric coating solution of Acryl-EZE (Colorcon) in purified water.

### Example 17

### Diltiazem-Losartan Press-Coated Tablets

According to the ingredients and contents shown in Table 3 below, diltiazem-losartan press-coated tablets were prepared in the same manner as in Example 16, except that volumes of the diltiazem layer and the losartan layer were increased.

### Example 18

### Diltiazem-Valsartan Press-Coated Tablets

According to the ingredients and contents shown in Table 3 below, diltiazem-valsartan press-coated tablets were prepared in the same manner as in Example 16, except that valsartan was used in place of losartan potassium, and calcium phosphate was used in place of lactose.

### Example 19

### Verapamil-Losartan Press-Coated Tablets

According to the ingredients and contents shown in Table 3 below, verapamil-losartan press-coated tablets were prepared in the same manner as in Example 16, except that verapamil hydrochloride and fumaric acid were used in place of diltiazem hydrochloride.

### Example 20

### Preparation of Losartan-Diltiazem Osmotic Press-Coated Tablets

(1) Preparation of Losartan Prior-Release Layer

The losartan prior-release layer was prepared in the same manner as in preparation of losartan-containing prior-release granules in Example 16.

(2) Diltiazem Osmotic Inner Core Tablets

According to the ingredients and contents shown in Table 3 below, diltiazem hydrochloride, microcrystalline cellulose and sodium chloride were sieved through a No. 35 sieve and mixed in a double cone mixer. Meanwhile, hydroxypropylcellulose was dissolved in purified water to prepare a binding solution. The mixture was placed in a high-speed mixer and the binding solution was added thereto, followed by kneading to prepare granules. The prepared granules were dried in a hot-water dryer at 60° C. and then sieved through a grinder equipped with a No. 25 sieve. The sieved material was placed in a double cone mixer and Carbomer 941 was added thereto, followed by mixing for 10 minutes. After completion of the mixing process, magnesium stearate was sieved through a No. 35 sieve and added to the mixture, followed by final mixing for 4 minutes to prepare diltiazem-containing delayed-release granules. The granules were compressed into tablets using a rotary tablet press (MRC-33: Sejong Machinery Co., Ltd., South Korea), thereby preparing diltiazem delayed-release inner core tablets. Thereafter, ethylcellulose and talc as insoluble coating bases were dispersed in purified water to prepare a dispersion which was then coated on the inner core in a Hi-coater (SFC-30N, Sejong Machinery Co., Ltd., South Korea), thereby preparing osmotic core tablets.

(3) Compression and Coating

Tablets having a hardness of 7 to 9 kp, a thickness of 6.0 mm and a diameter of 9.5 mm were prepared including the diltiazem osmotic core tablet as an inner core and the losartan-containing composition as an outer layer, using a press-coated tablet press (RUD-1: Kilian) at 30 rpm. Meanwhile, hydroxypropylmethylcellulose 2910, polyethylene glycol 6,000 and titanium oxide were dissolved in an ethanol-methylene chloride mixture to prepare a film coating solution. The film coating solution was coated on the compressed tablets in a Hi-coater (SFC-30N, Sejong Machinery Co., Ltd., South Korea) to form a coating layer, thereby preparing press-coated tablets.

### Example 21

### Preparation of Diltiazem-Losartan Capsules (Single Pellets)

(1) Preparation of Diltiazem-Containing Pellets

According to the ingredients and contents shown in Table 4 below, diltiazem hydrochloride, lactose, povidone (Kollidon CL, BASF), and talc were dissolved or dispersed in ethanol to prepare a drug coating solution. Sugar spheres (Non-pareil-101, Freund) were placed in a fluidized bed coater and the drug coating solution was sprayed thereon to prepare drug-containing pellets. Meanwhile, hydroxypropylmethylcellulose phthalate and Myvacet were dissolved in an ethanol/methylene chloride mixture to prepare a coating solution. The diltiazem drug-containing pellets were placed in a fluidized bed coater and then coated with the coating solution to prepare pellets.

(2) Preparation of Losartan-Containing Pellets

According to the ingredients and contents shown in Table 4 below, losartan potassium, lactose, and hydroxypropylmethylcellulose were dissolved in ethanol to prepare a solution which was then further coated on the diltiazem-containing pellets of Process (1) in a fluidized bed coater. Meanwhile, hydroxypropylmethylcellulose 2910, polyethylene glycol 6,000, and titanium oxide were dissolved in an ethanol/methylene chloride mixture to prepare a film coating solution which was then coated on the above-prepared pellets.

(3) Capsule Filling

The above-prepared pellets were filled in capsules using a capsule filling machine.

### Example 22

### Preparation of Diltiazem-Losartan Capsules (Pellets+Pellets)

(1) Preparation of Losartan-Containing Pellets

According to the ingredients and contents shown in Table 4 below, losartan potassium, lactose, and microcrystalline cellulose were placed in a fluidized bed coater, and a binding solution of hydroxypropylmethylcellulose in ethanol was sprayed thereon to prepare pellets. After preparation of pellets was complete, colloidal silicon dioxide was added thereto, followed by final mixing in a fluidized bed coater.

(2) Preparation of Diltiazem-Containing Pellets

Diltiazem-containing pellets were prepared in the same manner as in preparation of diltiazem-containing pellets in Process (1) of Example 21.

(3) Capsule Filling

The losartan-containing pellets of Process (1) and the diltiazem-containing pellets of Process (2) were filled in capsules using a capsule filling machine to prepare capsules.

### Example 23

### Preparation of Diltiazem-Losartan Capsules (Pellets+Granules)

(1) Preparation of Losartan-Containing Granules

According to the ingredients and contents shown in Table 4 below, losartan potassium, hydroxypropylmethylcellulose, and lactose were sieved through a No. 35 sieve, followed by granulation in a compression granulator (roller compactor). The prepared granules and pregelatinized starch were placed in a double cone mixer, followed by mixing for 15 minutes to prepare a mixture. After completion of the mixing process, sodium starch glycolate was added thereto, followed by further mixing for 8 minutes. Colloidal silicon dioxide (Aerosil 200VV, Degussa) was sieved through a No. 35 sieve and then added to the mixture, followed by final mixing for 4 minutes to prepare losartan prior-release granules.

(2) Preparation of Diltiazem-Containing Pellets

Diltiazem-containing pellets were prepared in the same manner as in preparation of diltiazem-containing pellets in Process (1) of Example 21.

(3) Capsule Filling

The losartan-containing granules of Process (1) and the diltiazem-containing pellets of Process (2) were filled in capsules using a capsule filling machine to prepare capsules.

### Example 24

### Preparation of Diltiazem-Losartan Capsules (Pellets+Tablets)

(1) Preparation of Losartan-Containing Tablets

According to the ingredients and contents shown in Table 4 below, losartan potassium, lactose, microcrystalline cellulose and pregelatinized starch were weighed and sieved through a No. 20 sieve and then mixed in a double cone mixer for 15 minutes to prepare a mixture. After completion of the mixing process, sodium starch glycolate was added thereto, followed by further mixing for 8 minutes. Magnesium stearate was sieved through a No. 35 sieve and then added to the mixture, followed by final mixing for 4 minutes to prepare granules. The granules were compressed into tablets. Meanwhile, hydroxypropylmethylcellulose and polyethylene glycol 6,000 were dissolved in ethanol to prepare a coating solution which was then coated on the compressed tablets.

(2) Preparation of Diltiazem-Containing Pellets

Diltiazem-containing pellets were prepared in the same manner as in preparation of diltiazem-containing pellets in Process (1) of Example 21.

(3) Capsule Filling

The losartan-containing tablets of Process (1) and the diltiazem-containing pellets of Process (2) were filled in capsules using a capsule filling machine to prepare capsules.

### Example 25

### Preparation of Diltiazem-Losartan Capsules (Granules+Granules)

(1) Preparation of Losartan Granules

Losartan granules were prepared in the same manner as in preparation of losartan-containing granules in Process (1) of Example 23.

(2) Preparation of Diltiazem-Containing Granules

According to the ingredients and contents shown in Table 4 below, diltiazem hydrochloride and microcrystalline cellulose were sieved through a No. 35 sieve and mixed in a double cone mixer for 5 minutes to prepare a mixture. Meanwhile, hydroxypropylcellulose was dissolved in purified water to prepare a binding solution. The mixture was placed in a fluidized bed granulator and kneaded with the binding solution to prepare granules, followed by drying. Meanwhile, hydroxypropylmethylcellulose phthalate and Myvacet (acetylated monoglyceride) were dissolved in an ethanol-methylene chloride mixture to prepare a coating solution. The dried material was placed in a fluidized bed coater and then coated with the coating solution. After completion of the coating process, magnesium stearate was added thereto, followed by mixing for 4 minutes to prepare diltiazem delayed-release granules.

(3) Capsule Filling

The above-prepared losartan granules and diltiazem-containing granules were filled in capsules using a capsule filling machine to prepare capsules.

### Example 26

### Preparation of Diltiazem-Losartan Capsules (Granules+Pellets)

(1) Preparation of Losartan-Containing Pellets

Losartan-containing pellets were prepared in the same manner as in preparation of losartan-containing pellets in Process (1) of Example 22.

(2) Preparation of Diltiazem-Containing Granules

Diltiazem-containing granules were prepared in the same manner as in preparation of diltiazem-containing granules in Process (2) of Example 25.

(3) Capsule Filling

The above-prepared losartan pellets and diltiazem-containing granules were filled in capsules using a capsule filling machine to prepare capsules.

### Example 27

### Preparation of Diltiazem-Losartan Capsules (Granules+Tablets)

(1) Preparation of Losartan-Containing Tablets

Losartan-containing tablets were prepared in the same manner as in preparation of losartan-containing tablets in Process (1) of Example 24.

(2) Preparation of Diltiazem-Containing Granules

Diltiazem-containing granules were prepared in the same manner as in preparation of diltiazem-containing granules in Process (2) of Example 25.

(3) Capsule Filling

The above-prepared losartan-containing tablets and diltiazem-containing granules were filled in capsules using a capsule filling machine to prepare capsules.

### Example 28

### Preparation of Diltiazem-Losartan Capsules (Tablets+Pellets)

(1) Preparation of Losartan-Containing Pellets

Losartan-containing pellets were prepared in the same manner as in preparation of losartan-containing pellets in Process (1) of Example 22.

(2) Preparation of Diltiazem-Containing Tablets

According to the ingredients and contents shown in Table 4 below, diltiazem hydrochloride and microcrystalline cellulose were sieved through a No. 35 sieve and mixed in a double cone mixer for 20 minutes. Meanwhile, hydroxypropylcellulose was dissolved in purified water to prepare a binding solution. The mixture was placed in a high-speed mixer and the binding solution was added thereto, followed by kneading to prepare granules. The prepared granules were dried in a hot-water dryer at 60° C. and then sieved through a grinder equipped with a No. 25 sieve. The sieved material was placed in a double cone mixer and Carbomer 941 was added thereto, followed by mixing for 10 minutes. After completion of the mixing process, magnesium stearate was sieved through a No. 35 sieve and then added to the mixture, followed by final mixing for 4 minutes to prepare diltiazem delayed-release granules. The granules were compressed into tablets using a rotary tablet press (MRC-33: Sejong Machinery Co., Ltd., South Korea), thereby preparing diltiazem tablets. Meanwhile, Acryl-EZE (Colorcon) was dissolved in purified water to prepare a coating solution which was then coated on the diltiazem tablets in a Hi-coater.

(3) Capsule Filling

The above-prepared diltiazem-containing tablets and losartan-containing pellets were placed in corresponding inlets of a capsule filling machine, followed by capsule filling.

### Example 29

### Preparation of Diltiazem-Losartan Capsules (Tablets+Granules)

(1) Preparation of Losartan Granules

Losartan granules were prepared in the same manner as in preparation of losartan-containing granules in Process (1) of Example 23.

(2) Preparation of Diltiazem-Containing Tablets

Diltiazem-containing tablets were prepared in the same manner as in preparation of diltiazem-containing tablets in Process (2) of Example 28.

(3) Capsule Filling

The above-prepared diltiazem-containing tablets and losartan-containing granules were placed in corresponding inlets of a capsule filling machine, followed by capsule filling.

### Example 30

### Preparation of Diltiazem-Losartan Capsules (Tablets+Tablets)

(1) Preparation of Losartan Tablets

Losartan tablets were prepared in the same manner as in preparation of losartan-containing tablets in Process (1) of Example 24.

(2) Preparation of Diltiazem-Containing Tablets

Diltiazem-containing tablets were prepared in the same manner as in preparation of diltiazem-containing tablets in Process (2) of Example 28.

(3) Capsule Filling

The above-prepared diltiazem-containing tablets and losartan-containing tablets were placed in corresponding inlets of a capsule filling machine, followed by capsule filling.

### Example 31

### Preparation of Diltiazem-Valsartan Capsules

According to the ingredients and contents shown in Table 5 below, diltiazem-valsartan capsules were prepared in the same manner as in Example 30, except that valsartan was used in place of losartan potassium, and calcium phosphate was used in place of lactose.

### Example 32

### Diltiazem-Telmisartan Double-Layered Tablets

According to the ingredients and contents shown in Table 5 below, diltiazem-telmisartan double-layered tablets were prepared in the same manner as in Example 30, except that telmisartan was used in place of losartan potassium, and sodium hydroxide was used in place of lactose.

### Example 33

### Diltiazem-Candesartan Double-Layered Tablets

According to the ingredients and contents shown in Table 5 below, diltiazem-candesartan double-layered tablets were prepared in the same manner as in Example 30, except that candesartan cilexetil was used in place of losartan potassium.

### Example 34

### Diltiazem-Irbesartan Double-Layered Tablets

According to the ingredients and contents shown in Table 5 below, diltiazem-irbesartan double-layered tablets were prepared in the same manner as in Example 30, except that irbesartan was used in place of losartan potassium.

### Example 35

### Diltiazem-Olmesartan Double-Layered Tablets

According to the ingredients and contents shown in Table 5 below, diltiazem-olmesartan double-layered tablets were prepared in the same manner as in Example 30, except that olmesartan medoxomil was used in place of losartan.

### Example 36

### Diltiazem-Eprosartan Double-Layered Tablets

According to the ingredients and contents shown in Table 5 below, diltiazem-eprosartan double-layered tablets were prepared in the same manner as in Example 30, except that eprosartan mesylate was used in place of losartan.

### Example 37

### Preparation of Verapamil-Losartan Capsules (Tablets+Tablets)

According to the ingredients and contents shown in Table 5 below, verapamil-losartan capsules were prepared in the same manner as in Example 30, except that verapamil hydrochloride and fumaric acid were used in place of diltiazem hydrochloride.

### Example 38

### Preparation of Diltiazem-Losartan Package Kits

According to the ingredients and contents shown in Table 5 below, diltiazem-losartan package kits were prepared in the same manner as in Example 30, except that the diltiazem-containing tablets and the losartan-containing tablets were packed in a PTP package container such that they can be simultaneously administered, in place of being filled in capsules.

### Example 39

### Preparation of Verapamil-Losartan Package Kits

According to the ingredients and contents shown in Table 5 below, verapamil-losartan package kits were prepared in the same manner as in Example 37, except that the verapamil-containing tablets and the losartan-containing tablets were packed in a PTP package container such that they can be simultaneously administered, in place of being filled in capsules.

TABLE 2

| | | Composition ratio (mg/tablet) | | | | | | | | | |
|---|---|---|---|---|---|---|---|---|---|---|---|
| | | | | | | | | | | | Exam- |
| Ingredients | | Example 1 | Example 2 | Example 3 | Example 4 | Example 5 | Example 6 | Example 7 | Example 8 | Example 9 | ple 10 |
| Prior- | Losartan | 50.0 | 50.0 | 50.0 | 50.0 | 50.0 | 100.0 | — | — | — | — |
| release | potassium | | | | | | | | | | |
| compartment | Valsartan | — | — | — | — | — | — | 80.0 | — | — | — |
| | Telmisartan | — | — | — | — | — | — | — | 40.0 | — | — |
| | Candesartan | — | — | — | — | — | — | — | — | 8.0 | — |
| | cilexetil | | | | | | | | | | |
| | Irbesartan | — | — | — | — | — | — | — | — | — | 150.0 |
| | Lactose | 50.0 | 50.0 | 15.0 | 50.0 | 50.0 | 100.0 | — | — | 50.0 | 50.0 |
| | Microcrystalline | 120.0 | 120.0 | — | 120.0 | 120.0 | 200.0 | 120.0 | 120.0 | 120.0 | 120.0 |
| | cellulose | | | | | | | | | | |
| | Pregelatinized | 20 | 20 | — | 20 | 20 | 40.0 | 20.0 | 20.0 | 20 | 20 |
| | starch | | | | | | | | | | |
| | Sodium | — | — | — | — | — | — | — | 50.0 | — | — |
| | hydroxide | | | | | | | | | | |
| | Calcium | — | — | — | — | — | — | 50.0 | — | — | — |
| | phosphate | | | | | | | | | | |
| | Hydroxypropyl | 5.0 | 5.0 | — | 5.0 | 5.0 | 10.0 | 5.0 | 5.0 | 5.0 | 5.0 |
| | cellulose | | | | | | | | | | |
| | Hydroxypropyl | — | — | 10.0 | — | — | — | — | — | — | — |
| | methylcellulose | | | | | | | | | | |
| | Sodium starch | 5.0 | 5.0 | — | 5.0 | 5.0 | 15.0 | 5.0 | 5.0 | 5.0 | 5.0 |
| | glycolate | | | | | | | | | | |
| | Magnesium | 2.0 | 2.0 | — | 2.0 | 2.0 | 5.0 | 2.0 | 2.0 | 2.0 | 2.0 |
| | stearate | | | | | | | | | | |
| | Talc | — | — | 5.0 | — | — | — | — | — | — | — |
| | Ethanol | — | — | 200.0 | — | — | — | — | — | — | — |
| | (volatile) | | | | | | | | | | |
| | Methylene | — | — | 200.0 | — | — | — | — | — | — | — |
| | chloride | | | | | | | | | | |
| | (volatile) | | | | | | | | | | |
| Total | | 252.0 | 252.0 | 80.0 | 252.0 | 252.0 | 470.0 | 282.0 | 242.0 | 210.0 | 352.0 |
| Delayed- | Diltiazem | 90.0 | 90.0 | 90.0 | 90.0 | 90.0 | 180.0 | 90.0 | 90.0 | 90 | 90 |
| release | hydrochloride | | | | | | | | | | |
| compartment | Verapamil | — | — | — | — | — | — | — | — | — | — |
| | hydrochloride | | | | | | | | | | |
| | Microcrystalline | 110.0 | 110.0 | 110.0 | 110.0 | 110.0 | 220.0 | 110.0 | 110.0 | 110.0 | 110.0 |
| | cellulose | | | | | | | | | | |
| | Sodium | — | — | — | — | — | — | — | — | — | — |
| | chloride | | | | | | | | | | |
| | Hydroxypropyl | 6.0 | 6.0 | 6.0 | 6.0 | 6.0 | 10.0 | 6.0 | 6.0 | 6.0 | 6.0 |
| | cellulose | | | | | | | | | | |
| | Carbomer 941 | — | — | 25.0 | — | 25.0 | 30.0 | 25.0 | 25.0 | 25.0 | 25.0 |
| | Ethylcellulose | 30.0 | 30.0 | — | 30.0 | — | — | — | — | — | — |
| | Hydroxypropyl | — | 15.0 | 20.0 | 15.0 | 20.0 | 30.0 | 20.0 | 20.0 | 20.0 | 20.0 |
| | methylcellulose | | | | | | | | | | |
| | phthalate | | | | | | | | | | |
| | Triethyl citrate | 3.0 | 3.0 | — | 3.0 | — | — | — | — | — | — |
| | Myvacet | — | — | 2.0 | — | — | — | — | — | — | — |
| | Magnesium | 1.0 | 1.0 | 2.0 | 1.0 | 2.0 | 4.0 | 2.0 | 2.0 | 2.0 | 2.0 |
| | stearate | | | | | | | | | | |
| | Purified water | 45.0 | 45.0 | 45.0 | 45.0 | 45.0 | 90.0 | 45.0 | 45.0 | 45.0 | 45.0 |
| | (volatile) | | | | | | | | | | |
| | Ethanol | 150.0 | 250.0 | 150.0 | 250.0 | 150.0 | 250.0 | 150.0 | 150.0 | 150.0 | 150.0 |
| | (volatile) | | | | | | | | | | |
| | Methylene | 150.0 | 250.0 | 150.0 | 250.0 | 150.0 | 250.0 | 150.0 | 150.0 | 150.0 | 150.0 |
| | chloride | | | | | | | | | | |
| | (volatile) | | | | | | | | | | |
| Total | | 240.0 | 255.0 | 255.0 | 255.0 | 253.0 | 474.0 | 253.0 | 253.0 | 253.0 | 253.0 |
| Coating | Hydroxypropyl | 9.0 | 9.0 | 9.0 | 9.0 | 9.0 | 9.0 | 9.0 | 9.0 | 9.0 | 9.0 |
| layer | methylcellulose | | | | | | | | | | |
| | 2910 | | | | | | | | | | |
| | Polyethylene | 0.7 | 0.7 | 1.0 | 0.7 | 0.7 | 0.7 | 0.7 | 0.7 | 0.7 | 0.7 |
| | glycol 6000 | | | | | | | | | | |
| | Titanium oxide | 0.3 | 0.3 | — | 0.3 | 0.3 | 0.3 | 0.3 | 0.3 | 0.3 | 0.3 |
| | Ethanol | 100.0 | 100.0 | 100.0 | 100.0 | 100.0 | 100.0 | 100.0 | 100.0 | 100.0 | 100.0 |
| | (volatile) | | | | | | | | | | |
| | Methylene | 100.0 | 100.0 | 100.0 | 100.0 | 100.0 | 100.0 | 100.0 | 100.0 | 100.0 | 100.0 |
| | chloride | | | | | | | | | | |
| | (volatile) | | | | | | | | | | |

| | | | | | | | | | | |
|---|---|---|---|---|---|---|---|---|---|---|
| Total | 502.0 | 517.0 | 345.0 | 517.0 | 515.0 | 954.0 | 545.0 | 505.0 | 473.0 | 615.0 |
| Oral formulation | Single | Single | Single | Double- | Double- | Double- | Double- | Double- | Double- | Double- |
| | tablet | tablet | tablet | layered | layered | layered | layered | layered | layered | layered |
| | | | | tablet | tablet | tablet | tablet | tablet | tablet | tablet |

TABLE 3

| | | Composition ratio (mg/tablet) | | | | | | | | | |
|---|---|---|---|---|---|---|---|---|---|---|---|
| | | Example | Example | Example | Example | Example | Example | Example | Example | Example | Exam- |
| Ingredients | | 11 | 12 | 13 | 14 | 15 | 16 | 17 | 18 | 19 | ple 20 |
| ARB drug | Losartan | — | — | 50.0 | 50.0 | 50.0 | 50.0 | 100.0 | — | 50.0 | 50.0 |
| | potassium | | | | | | | | | | |
| | Valsartan | — | — | — | — | — | — | — | 80.0 | — | — |
| | Olmesartan | 20.0 | — | — | — | — | — | — | — | — | — |
| | medoxomil | | | | | | | | | | |
| | Eprosartan | — | 500.0 | — | — | — | — | — | — | — | — |
| | mesylate | | | | | | | | | | |
| Excipient | Lactose | 50.0 | 50.0 | 50.0 | 50.0 | 100.0 | 100.0 | 100.0 | — | 100.0 | 100.0 |
| Excipient | Microcrystalline | 120.0 | 120.0 | 120.0 | 120.0 | 245.0 | 245.0 | 245.0 | 245.0 | 245.0 | 245.0 |
| | cellulose | | | | | | | | | | |
| Excipient | Pregelatinized | 20.0 | 20.0 | 20.0 | 20.0 | 65.0 | 65.0 | 65.0 | 65.0 | 65.0 | 65.0 |
| | starch | | | | | | | | | | |
| Excipient | Calcium | — | — | — | — | — | — | — | 100.0 | — | — |
| | phosphate | | | | | | | | | | |
| Binder | Hydroxypropyl- | 5.0 | 5.0 | 5.0 | 5.0 | 10.0 | 10.0 | 10.0 | 10.0 | 10.0 | 10.0 |
| | cellulose | | | | | | | | | | |
| Disintegrant | Sodium starch | 5.0 | 5.0 | 5.0 | 5.0 | 5.0 | 5.0 | 25.0 | 5.0 | 5.0 | 5.0 |
| | glycolate | | | | | | | | | | |
| Glidant | Magnesium | 2.0 | 2.0 | 2.0 | 2.0 | 5.0 | 5.0 | 5.0 | 5.0 | 5.0 | 5.0 |
| | stearate | | | | | | | | | | |
| Total | | 222.0 | 702.0 | 252.0 | 252.0 | 480.0 | 480.0 | 550.0 | 510.0 | 480.0 | 480.0 |
| Calcium | Diltiazem | 90.0 | 90.0 | — | 90.0 | 90.0 | 90.0 | 180.0 | 90.0 | — | 90.0 |
| antagonist | hydrochloride | | | | | | | | | | |
| | Verapamil | — | — | 120.0 | — | — | — | — | — | 120.0 | — |
| | hydrochloride | | | | | | | | | | |
| Excipient | Microcrystalline | 110.0 | 110.0 | 110.0 | 260.0 | 40.0 | 40.0 | 75.0 | 40.0 | 40.0 | 40.0 |
| | cellulose | | | | | | | | | | |
| Excipient | Fumaric acid | — | — | 30.0 | — | — | — | — | — | 30.0 | — |
| Osmo- | Sodium | — | — | — | — | — | — | — | — | — | 30.0 |
| regulator | chloride | | | | | | | | | | |
| Binder | Hydroxypropyl- | 6.0 | 6.0 | 6.0 | 6.0 | 4.0 | 4.0 | 8.0 | 4.0 | 4.0 | 4.0 |
| | cellulose | | | | | | | | | | |
| Water- | Carbomer 941 | 25.0 | 25.0 | 25.0 | 25.0 | 10.0 | 10.0 | 18.0 | 10.0 | 10.0 | 10.0 |
| insoluble | | | | | | | | | | | |
| polymer | | | | | | | | | | | |
| Water- | Ethylcellulose | — | — | — | — | — | — | — | — | — | 15.0 |
| insoluble | | | | | | | | | | | |
| polymer | | | | | | | | | | | |
| Enteric | Hydroxypropyl- | 20 | — | 20 | 20.0 | — | — | — | — | — | — |
| polymer | methylcellulose | | | | | | | | | | |
| | phthalate | | | | | | | | | | |
| Enteric | Acryl-EZE | — | — | — | — | — | 12.0 | 20.0 | 12.0 | 12.0 | — |
| coating | | | | | | | | | | | |
| base | | | | | | | | | | | |
| Glidant | Magnesium | 2.0 | 2.0 | 2.0 | 7.0 | 1.0 | 1.0 | 4.0 | 1.0 | 1.0 | 1.0 |
| | stearate | | | | | | | | | | |
| Glidant | Talc | — | — | — | — | — | — | — | — | — | 10 |
| Solvent | Purified water | 45.0 | 45.0 | 45.0 | 45.0 | 40.0 | 160.0 | 320.0 | 160.0 | 160.0 | 191.0 |
| | (volatile) | | | | | | | | | | |
| Solvent | Ethanol | 150.0 | 150.0 | 150.0 | 150.0 | — | — | — | — | — | — |
| | (volatile) | | | | | | | | | | |
| Solvent | Methylene | 150.0 | 150.0 | 150.0 | 150.0 | — | — | — | — | — | — |
| | chloride | | | | | | | | | | |
| | (volatile) | | | | | | | | | | |
| Total | | 253.0 | 233.0 | 313.0 | 408.0 | 145.0 | 157.0 | 305.0 | 157.0 | 217.0 | 191.0 |
| Film | Hydroxypropyl- | 9.0 | 9.0 | 9.0 | 9.0 | 9.0 | 9.0 | 9.0 | 9.0 | 9.0 | 9.0 |
| coating | methylcellulose | | | | | | | | | | |
| base | 2910 | | | | | | | | | | |
| Plasticizer | Polyethylene | 0.7 | 0.7 | 1.0 | 0.7 | 0.7 | 0.7 | 0.7 | 0.7 | 0.7 | 0.7 |
| | glycol 6000 | | | | | | | | | | |
| Colorant | Titanium | 0.3 | 0.3 | — | 0.3 | 0.3 | 0.3 | 0.3 | 0.3 | 0.3 | 0.3 |
| | oxide | | | | | | | | | | |
| Film | Ethanol | 100.0 | 100.0 | 100.0 | 100.0 | 100.0 | 100.0 | 100.0 | 100.0 | 100.0 | 100.0 |
| coating | (volatile) | | | | | | | | | | |
| solvent | | | | | | | | | | | |
| Film | Methylene | 100.0 | 100.0 | 100.0 | 100.0 | 100.0 | 100.0 | 100.0 | 100.0 | 100.0 | 100.0 |
| coating | chloride | | | | | | | | | | |
| solvent | (volatile) | | | | | | | | | | |

| | | | | | | | | | | |
|---|---|---|---|---|---|---|---|---|---|---|
| Total | 485.0 | 945.0 | 575.0 | 670.0 | 635.0 | 647.0 | 865.0 | 677.0 | 707.0 | 680.0 |
| Oral formulation | Double- | Double- | Double- | Triple- | Press- | Press- | Press- | Press- | Press- | Press- |
| | layered | layered | layered | layered | coated | coated | coated | coated | coated | coated |
| | tablet | tablet | tablet | tablet | tablet | tablet | tablet | tablet | tablet | tablet |

TABLE 4

| | | Composition ratio (mg/tablet) | | | | | | | | | |
|---|---|---|---|---|---|---|---|---|---|---|---|
| | | Example | Example | Example | Example | Example | Example | Example | Example | Example | Exam- |
| Ingredients | | 21 | 22 | 23 | 24 | 25 | 26 | 27 | 28 | 29 | ple 30 |
| Prior- | Losartan | 50.0 | 50.0 | 50.0 | 50.0 | 50.0 | 50.0 | 50.0 | 50.0 | 50.0 | 50.0 |
| release | potassium | | | | | | | | | | |
| compartment | Lactose | 20.0 | 20.0 | 50.0 | 50.0 | 50.0 | 20.0 | 50.0 | 20.0 | 50.0 | 50.0 |
| | Microcrystalline | — | 49.0 | — | 120.0 | — | — | 120.0 | — | — | 120.0 |
| | cellulose | | | | | | | | | | |
| | Pregelatinized | — | — | 20.0 | 20.0 | 20.0 | — | 20.0 | — | 20.0 | 20.0 |
| | starch | | | | | | | | | | |
| | Hydroxypropyl- | 10.0 | 10.0 | 20.0 | 6.0 | 20.0 | 10.0 | 6.0 | 10.0 | 20.0 | 6.0 |
| | methylcellulose | | | | | | | | | | |
| | Sodium | — | — | 3.0 | 5.0 | 3.0 | — | 5.0 | — | 3.0 | 5.0 |
| | starch | | | | | | | | | | |
| | glycolate | | | | | | | | | | |
| | Colloidal | — | 1.0 | 2.0 | — | 2.0 | 1.0 | — | 1.0 | 2.0 | — |
| | silicon | | | | | | | | | | |
| | dioxide | | | | | | | | | | |
| | Magnesium | — | — | — | 2.0 | — | — | 2.0 | — | — | 2.0 |
| | stearate | | | | | | | | | | |
| | Polyethylene | — | — | — | 1.0 | — | — | 1.0 | — | — | 1.0 |
| | glycol | | | | | | | | | | |
| | 6000 | | | | | | | | | | |
| | Ethanol | 300.0 | 45.0 | — | 60.0 | — | 300.0 | 60.0 | 300.0 | — | 60.0 |
| Total | | 80.0 | 130.0 | 145.0 | 254.0 | 145.0 | 81.0 | 254.0 | 81.0 | 145.0 | 254.0 |
| Delayed- | Diltiazem | 90.0 | 90.0 | 90.0 | 90.0 | 90.0 | 90.0 | 90.0 | 90.0 | 90.0 | 90.0 |
| release | hydrochloride | | | | | | | | | | |
| compartment | Verapamil | — | — | — | — | — | — | — | — | — | — |
| | hydrochloride | | | | | | | | | | |
| | Microcrystalline | — | — | — | — | 110.0 | 110.0 | 110.0 | 40.0 | 40.0 | 40.0 |
| | cellulose | | | | | | | | | | |
| | Lactose | 40.0 | 40.0 | 40.0 | 40.0 | — | — | — | — | — | — |
| | Sugar | 150.0 | 150.0 | 150.0 | 150.0 | — | — | — | — | — | — |
| | spheres | | | | | | | | | | |
| | Povidone | 13.0 | 13.0 | 13.0 | 13.0 | — | — | — | — | — | — |
| | Hydroxypropyl- | — | — | — | — | 6.0 | 6.0 | 6.0 | 4.0 | 4.0 | 4.0 |
| | cellulose | | | | | | | | | | |
| | Carbomer | — | — | — | — | — | — | — | 10.0 | 10.0 | 10.0 |
| | 941 | | | | | | | | | | |
| | Hydroxypropyl- | 10.0 | 10.0 | 10.0 | 10.0 | 20.0 | 20.0 | 20.0 | — | — | — |
| | methylcellulose | | | | | | | | | | |
| | phthalate | | | | | | | | | | |
| | Acryl-EZE | — | — | — | — | — | — | — | 12.0 | 12.0 | 12.0 |
| | Magnesium | — | — | — | — | 2.0 | 2.0 | 2.0 | 1.0 | 1.0 | 1.0 |
| | stearate | | | | | | | | | | |
| | Talc | 5.0 | 5.0 | 5.0 | 5.0 | — | — | — | — | — | — |
| | Myvacet | 1.0 | 1.0 | 1.0 | 1.0 | 2.0 | 2.0 | 2.0 | — | — | — |
| | Purified | — | — | — | — | 45.0 | 45.0 | 45.0 | 120.0 | 120.0 | 120.0 |
| | water | | | | | | | | | | |
| | (volatile) | | | | | | | | | | |
| | Ethanol | 650.0 | 650.0 | 650.0 | 650.0 | 150.0 | 150.0 | 150.0 | — | — | — |
| | (volatile) | | | | | | | | | | |
| | Methylene | 150.0 | 150.0 | 150.0 | 150.0 | 150.0 | 150.0 | 150.0 | — | — | — |
| | chloride | | | | | | | | | | |
| | (volatile) | | | | | | | | | | |
| Total | | 309.0 | 309.0 | 309.0 | 309.0 | 230.0 | 230.0 | 230.0 | 157.0 | 157.0 | 157.0 |
| Coating | Hydroxypropyl- | 9.0 | — | — | — | — | — | — | — | — | — |
| layer | methylcellulose | | | | | | | | | | |
| | 2910 | | | | | | | | | | |
| | Polyethylene | 0.7 | — | — | — | — | — | — | — | — | — |
| | glycol | | | | | | | | | | |
| | 6000 | | | | | | | | | | |
| | Titanium | 0.3 | — | — | — | — | — | — | — | — | — |
| | oxide | | | | | | | | | | |
| | Ethanol | 100.0 | — | — | — | — | — | — | — | — | — |
| | (volatile) | | | | | | | | | | |
| | Methylene | 100.0 | — | — | — | — | — | — | — | — | — |
| | chloride | | | | | | | | | | |
| | (volatile) | | | | | | | | | | |

| | | | | | | | | | | |
|---|---|---|---|---|---|---|---|---|---|---|
| Total | 399.0 | 390.0 | 454.0 | 563.0 | 375.0 | 311.0 | 484.0 | 238.0 | 302.0 | 411.0 |
| Oral formulation | Capsule | Capsule | Capsule | Capsule | Capsule | Capsule | Capsule | Capsule | Capsule | Capsule |

TABLE 5

| | | Composition ratio (mg/tablet) | | | | | | | | |
|---|---|---|---|---|---|---|---|---|---|---|
| | | Example | Example | Example | Example | Example | Example | Example | Example | Example |
| Ingredients | | 31 | 32 | 33 | 34 | 35 | 36 | 37 | 38 | 39 |
| Prior- | Losartan | — | — | — | — | — | — | 50.0 | 50.0 | 50.0 |
| release | potassium | | | | | | | | | |
| compartment | Valsartan | 80.0 | — | — | — | — | — | — | — | — |
| | Telmisartan | — | 40.0 | — | — | — | — | — | — | — |
| | Candesartan | — | — | 8.0 | — | — | — | — | — | — |
| | cilexetil | | | | | | | | | |
| | Irbesartan | — | — | — | 150.0 | — | — | — | — | — |
| | Olmesartan | — | — | — | — | 20.0 | — | — | — | — |
| | medoxomil | | | | | | | | | |
| | Eprosartan | — | — | — | — | — | 500.0 | — | — | — |
| | mesylate | | | | | | | | | |
| | Lactose | — | — | 50.0 | 50.0 | 50.0 | 50.0 | 50.0 | 50.0 | 50.0 |
| | Microcrystalline | 120.0 | 120.0 | 120.0 | 120.0 | 120.0 | 120.0 | 120.0 | 120.0 | 120.0 |
| | cellulose | | | | | | | | | |
| | Pregelatinized | 20.0 | 20.0 | 20.0 | 20.0 | 20.0 | 20.0 | 20.0 | 20.0 | 20.0 |
| | starch | | | | | | | | | |
| | Calcium | 50.0 | — | — | — | — | — | — | — | — |
| | phosphate | | | | | | | | | |
| | Sodium | — | 50.0 | — | — | — | — | — | — | — |
| | hydroxide | | | | | | | | | |
| | Hydroxypropyl- | 6.0 | 6.0 | 6.0 | 6.0 | 6.0 | 6.0 | 6.0 | 6.0 | 6.0 |
| | methylcellulose | | | | | | | | | |
| | Sodium starch | 5.0 | 5.0 | 5.0 | 5.0 | 5.0 | 5.0 | 5.0 | 5.0 | 5.0 |
| | glycolate | | | | | | | | | |
| | Magnesium | 2.0 | 2.0 | 2.0 | 2.0 | 2.0 | 2.0 | 2.0 | 2.0 | 2.0 |
| | stearate | | | | | | | | | |
| | Polyethylene | 1.0 | 1.0 | 1.0 | 1.0 | 1.0 | 1.0 | 1.0 | 1.0 | 1.0 |
| | glycol | | | | | | | | | |
| | 6000 | | | | | | | | | |
| | Ethanol | 60.0 | 60.0 | 60.0 | 60.0 | 60.0 | 60.0 | 60.0 | 60.0 | 60.0 |
| Total | | 284.0 | 244.0 | 212.0 | 354.0 | 224.0 | 704.0 | 254.0 | 254.0 | 254.0 |
| Delayed- | Diltiazem | 90.0 | 90.0 | 90.0 | 90.0 | 90.0 | 90.0 | — | 90.0 | — |
| release | hydrochloride | | | | | | | | | |
| compartment | Verapamil | — | — | — | — | — | — | 120.0 | — | 120.0 |
| | hydrochloride | | | | | | | | | |
| | Microcrystalline | 40.0 | 40.0 | 40.0 | 40.0 | 40.0 | 40.0 | 40.0 | 40.0 | 40.0 |
| | cellulose | | | | | | | | | |
| | Fumaric acid | — | — | — | — | — | — | 30.0 | — | 30.0 |
| | Hydroxypropyl- | 4.0 | 4.0 | 4.0 | 4.0 | 4.0 | 4.0 | 4.0 | 4.0 | 4.0 |
| | cellulose | | | | | | | | | |
| | Carbomer 941 | 10.0 | 10.0 | 10.0 | 10.0 | 10.0 | 10.0 | 10.0 | 10.0 | 10.0 |
| | Acryl-EZE | 12.0 | 12.0 | 12.0 | 12.0 | 12.0 | 12.0 | 12.0 | 12.0 | 12.0 |
| | Magnesium | 1.0 | 1.0 | 1.0 | 1.0 | 1.0 | 1.0 | 1.0 | 1.0 | 1.0 |
| | stearate | | | | | | | | | |
| | Purified water | 120.0 | 120.0 | 120.0 | 120.0 | 120.0 | 120.0 | 120.0 | 120.0 | 120.0 |
| | (volatile) | | | | | | | | | |
| | Ethanol (volatile) | — | — | — | — | — | — | — | — | — |
| | Methylene | — | — | — | — | — | — | — | — | — |
| | chloride | | | | | | | | | |
| | (volatile) | | | | | | | | | |

| | | | | | | | | | |
|---|---|---|---|---|---|---|---|---|---|
| Total | 157.0 | 157.0 | 157.0 | 157.0 | 157.0 | 157.0 | 217.0 | 157.0 | 217.0 |
| Sum total | 441.0 | 401.0 | 369.0 | 511.0 | 381.0 | 861.0 | 471.0 | 411.0 | 471.0 |
| Oral Formulation | Capsule | Capsule | Capsule | Capsule | Capsule | Capsule | Capsule | Kit formulation | Kit formulation |

### Experimental Example 1

### Dissolution Profile Test

A dissolution profile test was performed using pharmaceutical formulations obtained in above-stated Examples, based on the general dissolution test method described in the Korean Pharmacopoeia (9^{th} revision).

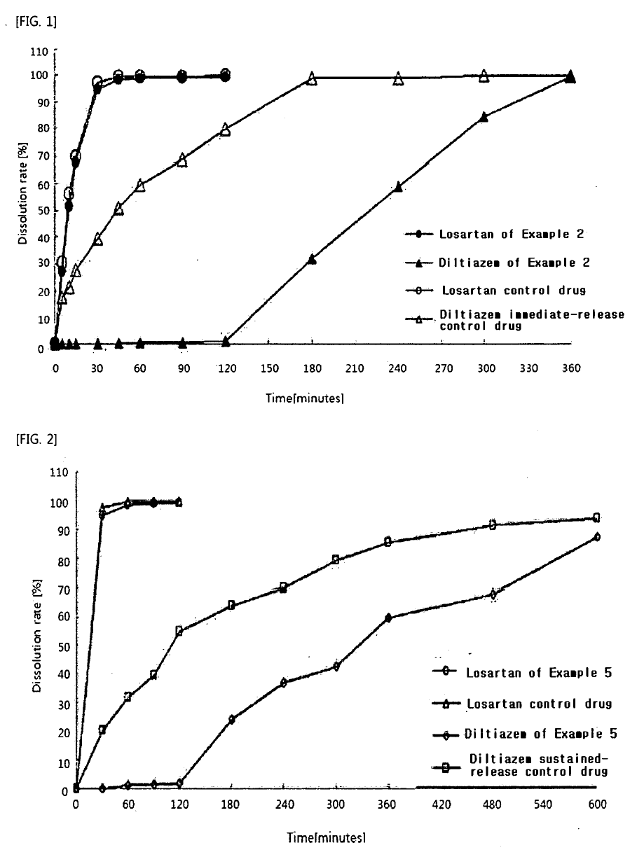

In a specific test method, a dissolution profile test was performed in 750 mL of a 0.1N hydrochloric acid solution warmed to 37±0.5° C. for 2 hours and then in 1,000 mL of a pH 6.8 simulated intestinal juice (second fluid in a disintegration test method described in the Korean Pharmacopoeia (8^{th} revision)). The dissolution medium was placed in a basket and a dissolution test was performed according to a paddle method at a paddle rotation speed of 50 rpm. For a formulation containing a poorly-soluble drug, the experiment was performed with the addition of a solubilizer polysorbate 80 or sodium lauryl sulfate for the discrimination of dissolution profiles. After the start of dissolution, a given amount of the dissolution medium was taken at a given interval of time and analyzed to measure the dissolution rate. The measurement results are given in FIGS. 1 to 6 (the number of test samples are 12, respectively).

As can be seen from the results of FIGS. 1 and 2, when the pharmaceutical formulation of non-dihydropyridine calcium channel blocker-ARB in accordance with the present invention was subjected to a dissolution profile test under the conditions of Experimental Example, an ARB drug, losartan was released first under acidic conditions (simulated gastric juice) immediately after the start of the dissolution profile test, similar to commercially available control drugs, whereas a non-dihydropyridine calcium channel blocker, diltiazem started to be released 2 to 4 hours after the start of the dissolution profile test, which corresponds to an intended time period of the present invention, unlike commercially available control drugs.

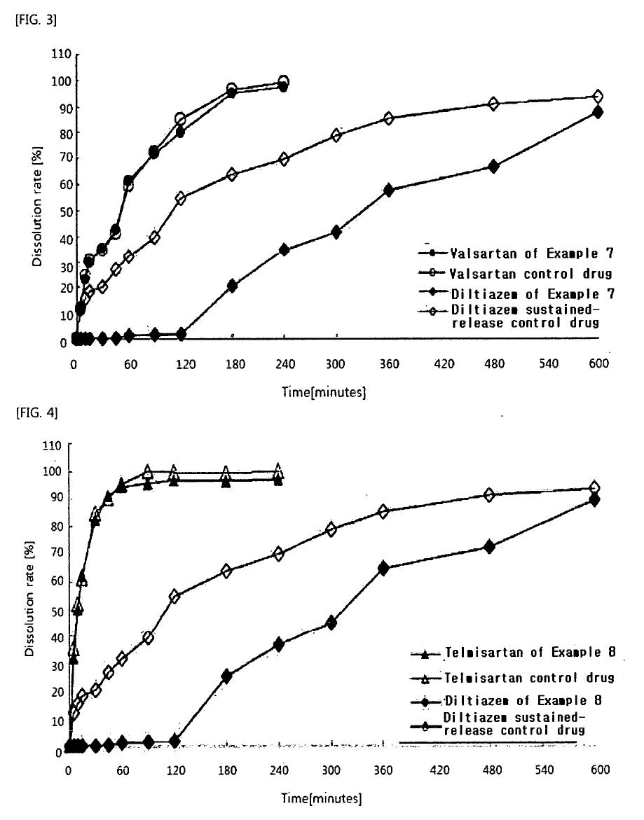

As can be seen from the results of FIGS. 3 and 4, even when the pharmaceutical formulation of the present invention was prepared using a different ARB drug, valsartan or telmisartan other than losartan, the ARB drug was first released with exhibiting no effect on release of the non-dihydropyridine calcium channel blocker.

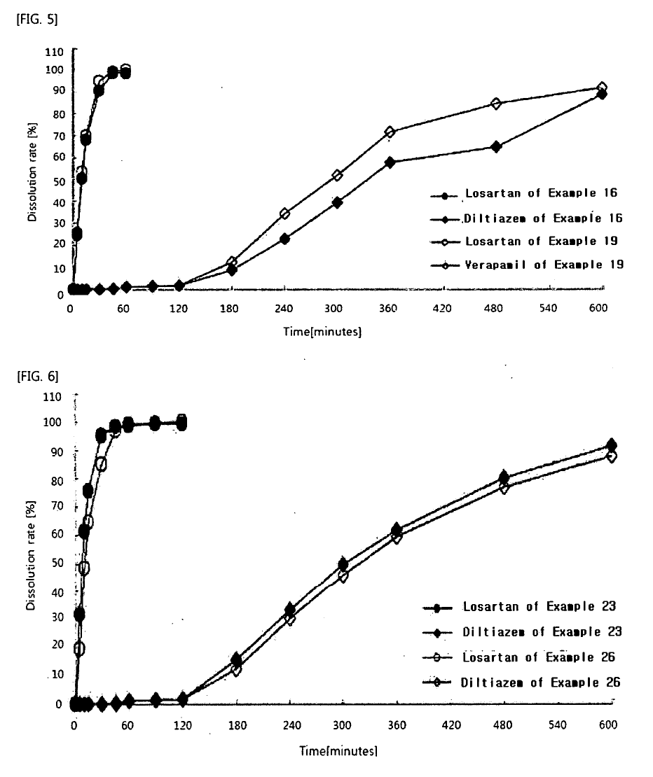

As can be seen from the results of FIGS. 5 and 6, even when the pharmaceutical formulation of the present invention was prepared into various dosage forms such as press-coated tablet and capsule, or a different non-dihydropyridine calcium channel blocker such as verapamil was used, dissolution patterns intended by the present invention were obtained.

### INDUSTRIAL APPLICABILITY

The pharmaceutical formulation of the present invention is a pharmaceutical formulation containing a non-dihydropyridine calcium channel blocker and ARB, which is designed based on Xenobiotics and Chronotherapy, and exhibits reduction of side effects due to co-administration of single drugs and avoidance of drug-drug interaction, in conjunction with high therapeutic effectiveness in treatment of hypertension and hyperlipidemia and prevention of complications in people with metabolic syndromes and improved patents' compliance and optimization of a drug delivery time.

1. A pharmaceutical formulation comprising a prior-release compartment containing an angiotensin-2 receptor blocker (ARB) as a pharmacologically active ingredient and a delayed-release compartment containing a non-dihydropyridine calcium channel blocker as a pharmacologically active ingredient.

2. The pharmaceutical formulation of claim 1, wherein the ARB is at least one selected from losartan, valsartan, telmisartan, eprosartan, irbesartan, candesartan, olmesartan, isomers thereof, pharmaceutically acceptable salts thereof, and prodrugs thereof.

3. The pharmaceutical formulation of claim 1, wherein the non-dihydropyridine calcium channel blocker is a non-dihydropyridine calcium channel blocker which inhibits the production of a cytochrome P450 enzyme and is at least one selected from among diltiazem, verapamil, gallopamil, cinnarizine, flunarizine, optical isomers thereof and pharmaceutically acceptable salts thereof.

4.-5. (canceled)

6. The pharmaceutical formulation of claim 1, wherein the ARB is at least one selected from among losartan, valsartan, telmisartan, candesartan, irbesartan, olmesartan, eprosartan, isomers thereof, and pharmaceutically acceptable salts thereof, and the non-dihydropyridine calcium channel blocker is at least one selected from among diltiazem, verapamil, isomers thereof and pharmaceutically acceptable salts thereof.

7.-13. (canceled)

14. The pharmaceutical formulation of claim 1, wherein the ARB is released at a level of more than 60% of a total amount of ARB in the formulation within one hour and the non-dihydropyridine calcium channel blocker is released at a level of less than 60% of a total amount of a non-dihydropyridine calcium channel blocker in the unit formulation up to 4 hours after initiation of the release of the ARB and the release of the non-dihydropyridine calcium channel blocker is initiated 2 hours after the release of the ARB is initiated and is finished within 24 hours.

15.-18. (canceled)

19. The pharmaceutical formulation of claim 1, wherein the delayed-release compartment contains at least one release-controlling material selected from among an enteric polymer, a water-insoluble polymer, a hydrophobic compound and a hydrophilic polymer.

20.-21. (canceled)

22. The pharmaceutical formulation of claim 19, wherein the enteric polymer is at least one selected from among an enteric cellulose derivative, an enteric acrylic acid copolymer, an enteric maleic acid copolymer and an enteric polyvinyl derivative, wherein:
the enteric cellulose derivative is at least one selected from among hydroxypropylmethylcellulose acetate succinate, hydroxypropylmethylcellulose phthalate, hydroxymethylethylcellulose phthalate, cellulose acetate phthalate, cellulose acetate succinate, cellulose acetate maleate, cellulose benzoate phthalate, cellulose propionate phthalate, methylcellulose phthalate, carboxymethylethylcellulose, ethylhydroxyethylcellulose phthalate, and methylhydroxyethylcellulose;
the enteric acrylic acid copolymer is at least one selected from among a styrene/acrylic acid copolymer, a methyl acrylate/acrylic acid copolymer, a methyl acrylate/methacrylic acid copolymer, a butyl acrylate/styrene/acrylic acid copolymer, a methacrylic acid/methyl methacrylate copolymer, a methacrylic acid/ethyl acrylate copolymer, and a methyl acrylate/methacrylic acid/octyl acrylate copolymer;
the enteric maleic acid copolymer is at least one selected from a vinylacetate/maleic anhydride copolymer, a styrene/maleic anhydride copolymer, a styrene/maleic monoester copolymer, a vinyl methyl ether/maleic anhydride copolymer, an ethylene/maleic anhydride copolymer, a vinyl butyl ether/maleic anhydride copolymer, an acrylonitrile/methyl acrylate/maleic anhydride copolymer, and a butyl acrylate/styrene/maleic anhydride copolymer; and
the enteric polyvinyl derivative is at least one selected from polyvinylalcohol phthalate, polyvinylacetal phthalate, polyvinylbutyrate phthalate, and polyvinylacetacetal phthalate.

23.-25. (canceled)

26. The pharmaceutical formulation of claim 19, wherein the water-insoluble polymer is at least one selected from among polyvinyl acetate, a water-insoluble polymethacrylate copolymer, ethylcellulose, cellulose ester, cellulose ether, cellulose acylate, cellulose diacylate, cellulose triacylate, cellulose acetate, cellulose diacetate, and cellulose triacetate.

27.-28. (canceled)

29. The pharmaceutical formulation of claim 19, wherein the hydrophobic compound is at least one selected from among a fatty acid or fatty acid ester, a fatty acid alcohol, a wax, and an inorganic material, and
the fatty acid or fatty acid ester is at least one selected from among glyceryl palmitostearate, glyceryl stearate, glyceryl behenate, cetyl palmitate, glyceryl monooleate and stearic acid;
the fatty acid alcohol is at least one selected from among cetostearyl alcohol, cetyl alcohol and stearyl alcohol;
the wax is at least one selected from among carnauba wax, beeswax and microcrystalline wax; and
the inorganic material is at least one selected from among talc, precipitated calcium carbonate, calcium hydrogen phosphate, zinc oxide, titanium oxide, kaolin, bentonite, montmorillonite and veegum.

30.-31. (canceled)

32. The pharmaceutical formulation of claim 19, wherein the hydrophilic polymer is at least one selected from a saccharide, a cellulose derivative, a gum, a protein, a polyvinyl derivative, a hydrophilic polymethacrylate copolymer, a polyethylene derivative, and a carboxyvinyl copolymer, wherein:
the saccharide is at least one selected from among dextrin, polydextrin, dextran, pectin and a pectin derivative, alginate, polygalacturonic acid, xylan, arabinoxylan, arabinogalactan, starch, hydroxypropyl starch, amylase and amylopectin;
the cellulose derivative is at least one selected from among hydroxypropylmethylcellulose, hydroxypropylcellulose, hydroxymethylcellulose, hydroxyethylcellulose, methylcellulose, sodium carboxymethylcellulose and hydroxyethylmethylcellulose;
the gum is at least one selected from among guar gum, locust bean gum, tragacanth, carrageenan, gum acacia, gum arabic, gellan gum and xanthan gum;
the protein is at least one selected from among gelatin, casein and zein;
the polyvinyl derivative is at least one selected from among polyvinyl alcohol, polyvinyl pyrrolidone and polyvinylacetal diethylaminoacetate;
the hydrophilic polymethacrylate copolymer is at least one selected from among a poly(butyl methacrylate, (2-dimethylaminoethyl)methacrylate, methyl methacrylate) copolymer, a poly(methacrylate, methyl methacrylate) copolymer and a poly(methacrylate, ethyl acrylate) copolymer;
the polyethylene derivative is at least one selected from among polyethylene glycol and polyethylene oxide; and
the carboxyvinyl polymer is carbomer.

33.-34. (canceled)

35. The pharmaceutical formulation of claim 1, wherein the pharmaceutical formulation is in the form of a two-phase matrix tablet including a delayed-release compartment and a prior-release compartment enclosing the delayed-release compartment.

36. The pharmaceutical formulation of claim 1, wherein the pharmaceutical formulation is in the form of a film-coated tablet including a tablet of a delayed-release compartment and a film-coating layer of a prior-release compartment enclosing the exterior of the tablet.

37. The pharmaceutical formulation of claim 1, wherein the pharmaceutical formulation is in the form of a multi-layered tablet having a multi-layered structure of the delayed-release compartment and the prior-release compartment.

38. The pharmaceutical formulation of claim 1, wherein the formulation is in the form of a press-coated tablet including an inner core tablet of a delayed-release compartment and an outer layer of a prior-release compartment enclosing the outer surface of the inner core tablet.

39. (canceled)

40. The pharmaceutical formulation of claim 1, wherein the pharmaceutical formulation is in the form of a capsule including a particle, granule, pellet, or tablet of a delayed-release compartment and a particle, granule, pellet, or tablet of a prior-release compartment.

41. The pharmaceutical formulation of claim 1, further comprising a coating layer on the outside of the delayed-release compartment and/or the prior-release compartment.

42. The pharmaceutical formulation of claim 1, wherein the delayed-release compartment:
contains an osmo-regulator which is at least one selected from among magnesium sulfate, magnesium chloride, sodium chloride, lithium chloride, potassium sulfate, sodium sulfate and lithium sulfate; and
is coated by a semi-permeable membrane coating base which is at least one selected from among polyvinyl acetate, a polymethacrylate copolymer, ethylcellulose, cellulose ester, cellulose ether, cellulose acylate, cellulose diacylate, cellulose triacylate, cellulose acetate, cellulose diacetate and cellulose triacetate.

43.-44. (canceled)

45. The pharmaceutical formulation of claim 1, wherein the pharmaceutical formulation is in the form of a coated tablet further including a coating layer on the outside thereof.

46. (canceled)

47. The pharmaceutical formulation of claim 1, wherein the formulation is for administration between 5:00 p.m. to 11:00 p.m.

48. A method for treating a cardiovascular disease, comprising administering a pharmaceutical formulation including a prior-release compartment containing an angiotensin-2 receptor blocker (ARB) as a pharmacologically active ingredient and a delayed-release compartment containing a non-dihydropyridine calcium channel blocker as a pharmacologically active ingredient to a mammal.

49.-50. (canceled)
